(19) United States
(12) Patent Application Publication
(10) Pub. No.: US 2004/0018955 A1
(43) Pub. Date: Jan. 29, 2004

(54) Pro-perfume composition

(76) Inventors: Jean Wevers, Steenhuffel (BE); Johan Smets, Lubbeek (BE); Rafael Trujillo Rosaldo, Mason, OH (US); Pascale Claire Annick Van Steenwinckel, Weerde (BE)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/168,936

(22) PCT Filed: Jan. 10, 2001
(86) PCT No.: PCT/US01/00822

(30) Foreign Application Priority Data
Jan. 12, 2000 (EP) 00870004.9
Apr. 13, 2000 (EP) 08870069.2

(51) Int. Cl. A61K 7/46

(52) U.S. Cl. 512/27

(57) ABSTRACT

There is provided a pro-perfume composition comprising an amino-functional component and a benefit agent, which is stably suspended into a liquid detergent matrix, and subsequently providing an enhanced deposition as well as long lasting release on the treated fabric of the benefit agent.

### TECHNICAL FIELD OF THE INVENTION

The present invention relates to a pro-perfume composition, in particular for use in liquid detergent composition for imparting sustained release of the benefit agent on the treated surfaces like fabrics, in particular dry fabrics.

### BACKGROUND OF THE INVENTION

Perfumed products are well-known in the art. However, consumer acceptance of such perfumed products like laundry and cleaning products is determined not only by the performance achieved with these products but also by the aesthetics associated therewith. The perfume components are therefore an important aspect of the successful formulation of such commercial products.

It is also desired by consumers for treated surfaces like fabrics to maintain the pleasing fragrance over time. Indeed, perfume additives make such compositions more aesthetically pleasing to the consumer, and in some cases the perfume imparts a pleasant fragrance to surfaces, like fabrics, treated therewith. However, the amount of perfume carried-over from an aqueous laundry or cleaning bath onto fabrics is often marginal and does not last long on the surface. Furthermore, fragrance materials are often very costly and their inefficient use in laundry and cleaning compositions and ineffective delivery to surfaces like fabrics results in a very high cost to both consumers and laundry and cleaning manufacturers. Industry, therefore, continues to seek with urgency for more efficient and effective fragrance delivery in laundry and cleaning products, especially for improvement in the provision of long-lasting fragrance to the surfaces like fabrics.

Recently, a new class of materials, namely the amine reaction product of a compound containing a primary amine functional group and an active ketone or aldehyde containing component, have found increasing use in the domestic treatment of fabrics in order to provide long lasting perfume release on the laundered fabric. Disclosure of such compounds can be found in recently filed applications EP 98870227.0, EP 98870226.2, EP 99870026.4, and EP 99870025.6, all incorporated herein by reference.

However, notwithstanding the advances in the art, there is still a need for improving the incorporation of pro-perfumes into detergent compositions, in particular liquid detergent compositions like heavy duty liquids, especially from a stability aspect.

Accordingly, it is an object of the present invention to provide a pro-perfume composition that is effectively suspended, especially for further incorporation into liquid detergent compositions.

It has now been found that the use of a suspension material like silicone which is in close physical proximity with the pro-perfume composition fulfills such a need.

### SUMMARY OF THE INVENTION

The present invention is a pro-perfume composition comprising an amino-functional polymer and a benefit agent, characterised in that the pro-perfume composition and the suspending material are in close physical proximity.

For the purpose of the present invention, the term “close physical proximity” means one of the following:

i)—a droplet in which said pro-perfume and said suspending material are in intimate admixture;

ii)—a droplet in which the pro-perfume is coated with one or more layers wherein at least one layer contains the suspending material.

It has to be understood by “close physical proximity” that the pro-perfume and the suspending material are not two separate discrete particles in the detergent composition.

### DETAILED DESCRIPTION OF THE INVENTION

An essential feature of the invention is a pro-perfume.

Pro-Perfume

Pro-perfume compounds for use in the present invention typically comprises an amino-functional component and a benefit agent.

A-Amino-Functional Component

Examples of amino-functional component are those which have chemically reacted with a benefit agent, so-called “amines which form amine reaction products”, i.e. a product of reaction between a compound containing a primary amine functional group and/or secondary amine functional group and an active ketone or aldehyde containing component. Preferred compounds for use herein are polymers which have been previously reacted with an aldehyde and/or ketone perfume ingredient, thereby imparting a more effective scent to the fabrics.

A typical disclosure of amine reaction product suitable for use herein can be found in recently filed applications EP 98870227.0, EP 98870226.2, EP 99870026.4, and EP 99870025.6, all incorporated herein by reference.

A-Primary Amine and/or Secondary Amine

By “primary and/or secondary amine”, it is meant a component which carries at least one primary and/or secondary amine and/or amide function.

Of course, one amine compound may carry both primary and secondary amine compound, thereby enabling the reaction with several aldehydes and/or ketones.

Preferably, the primary amine and/or secondary amine compound is also characterized by an Odour Intensity Index of less than that of a 1% solution of methylanthranilate in dipropylene glycol.

Odour Intensity Index Method

By Odour Intensity Index, it meant that the pure chemicals were diluted at 1% in Dipropylene Glycol, odor-free solvent used in perfumery. This percentage is more representative of usage levels. Smelling strips, or so called “blotters”, were dipped and presented to the expert panellist for evaluation. Expert panellists are assessors trained for at least six months in odor grading and whose gradings are checked for accuracy and reproducibility versus a reference on an on-going basis. For each amine compound, the panellist was presented two blotters: one reference (Me Anthranilate, unknown from the panellist) and the sample. The panellist was asked to rank both smelling strips on the 0-5 odor intensity scale, 0 being no odor detected, 5 being very strong odor present.

Results:

The following represents Odour Intensity Index of an amine compound suitable for use in the present invention and according to the above procedure. In each case, numbers are arithmetic averages among 5 expert panellists and the results are statistically significantly different at 95% confidence level:

| | |
|---|---|
| Methylanthranilate 1% (reference) | 3.4 |
| Ethyl-4-aminobenzoate (EAB) 1% | 0.9 |
| 1,4-bis-(3-aminopropyl)-piperazine (BNPP) 1% | 1.0 |

A general structure for the primary amine compound of the invention is as follows:

B—(NH2)ₙ;

wherein B is a carrier material, and n is an index of value of at least 1.

Compounds containing a secondary amine group have a structure similar to the above excepted that the compound comprises one or more —NH— groups instead of —NH2. Further, the compound structure may also have one or more of both —NH2 and —NH— groups.

Preferred B carriers are inorganic or organic carriers.

By “inorganic carrier”, it is meant carrier which are non-or substantially non carbon based backbones.

Preferred primary and/or secondary amines, among the inorganic carriers, are those selected from mono or polymers or organic-organosilicon copolymers of amino derivatised organo silane, siloxane, silazane, alumane, aluminum siloxane, or aluminum silicate compounds. Typical examples of such carriers are: organosiloxanes with at least one primary amine moiety like the diaminoalkylsiloxane [H2NCH2(CH3) 2Si]O, or the organoaminosilane (C6H5) 3SiNH2 described in: Chemistry and Technology of Silicone, W. Noll, Academic Press Inc. 1998, London, pp 209, 106).

Preferred primary and/or secondary amines, among the organic carriers, are those selected from aminoaryl derivatives, polyamines, amino acids and derivatives thereof, substituted amines and amides, glucamines, dendrimers, polyvinylamines and derivatives thereof, and/or copolymer thereof, alkylene polyamine, polyaminoacid and copolymer thereof, cross-linked polyaminoacids, amino substituted polyvinylalcohol, polyoxyethylene bis amine or bis aminoalkyl, aminoalkyl piperazine and derivatives thereof, bis (amino alkyl) alkyl diamine linear or branched, and mixtures thereof.

Preferred aminoaryl derivatives are the amino-benzene derivatives including the alkyl esters of 4-amino benzoate compounds, and more preferably selected from ethyl-4-amino benzoate, phenylethyl-4-aminobenzoate, phenyl-4-aminobenzoate, 4-amino-N′-(3-aminopropyl)-benzamide, and mixtures thereof.

Polyamines suitable for use in the present invention are polyethyleneimines polymers, poly[oxy(methyl-1,2-ethanediyl)], α-(2-aminomethylethyl)-ω-(2-aminomethyl-ethoxy)-(=C.A.S No. 9046-10-0); poly[oxy(methyl-1,2-ethanediyl)], α-hydro-)-ω-(2-aminomethylethoxy)-, ether with 2-ethyl-2-(hydroxymethyl)-1,3-propanediol (=C.A.S. No. 39423-51-3); commercially available under the tradename Jeffamines T-403, D-230, D-400, D-2000; 2,2′,2″-triaminotriethylamine; 2,2′-diamino-diethylamine; 3,3′-diamino-dipropylamine, 1,3 bis aminoethyl-cyclohexane commercially available from Mitsubishi and the C12 Sternamines commercially available from Clariant like the C12 Sternamin(propylenamine)ₙ with n=3/4, and mixtures thereof. Preferred polyamines are polyethyleneimines commercially available under the tradename Lupasol like Lupasol FG (MW 800), G20wfv (MW 1300), PR8515 (MW 2000), WF (MW 25000), FC (MW 800), G20 (MW 1300), G35 (MW 1200), G100 (MW 2000), HF (MW 25000), P (MW 750000), PS (MW 750000), SK (MW 2000000), SNA (MW 1000000), most preferably Lupasol HF or WF (MW 25000), P (MW 750000), PS (MW 750000), SK (MW 2000000), SNA (MW 1000000).

Preferred amino acids for use herein are selected from tyrosine, tryptophane, lysine, glutamic acid, glutamine, aspartic acid, arginine, asparagine, phenylalanine, proline, glycine, serine, histidine, threonine, methionine, and mixture thereof, most preferably selected from tyrosine, tryptophane, and mixture thereof. Preferred amino acid derivatives are selected from tyrosine ethylate, glycine methylate, tryptophane ethylate, and mixture thereof.

Preferred substituted amines and amides for use herein are selected from nipecotamide, N-coco-1,3-propenediamine; N-oleyl-1,3-propenediamine; N-(tallow alkyl)-1,3-propenediamine; 1,4-diamino cyclohexane; 1,2-diamino-cyclohexane; 1,12-diaminododecane, and mixtures thereof.

Other primary amine compounds suitable for use herein are the glucamines, preferably selected from 2,3,4,5,6-pentamethoxy-glucamine; 6-acetylglucamine, glucamine, and mixture thereof.

Also preferred compounds are the polyethylenimine and/or polypropylenimine dendrimers and the commercially available Starburst® polyamidoamines (PAMAM) dendrimers, generation G0-G10 from Dendritech and the dendrimers Astromols®, generation 1-5 from DSM being DiAminoButane PolyAmine DAB (PA)x dendrimers with x=2ⁿ×4 and n being generally comprised between 0 and 4.

Polyamino acid is one suitable and preferred class of amino-functional polymer. Polyaminoacids are compounds which are made up of amino acids or chemically modified amino acids. They can contain alanine, serine, aspartic acid, arginine, valine, threonine, glutamic acid, leucine, cysteine, histidine, lysine, isoleucine, tyrosine, asparagine, methionine, proline, tryptophan, phenylalanine, glutamine, glycine or mixtures thereof. In chemically modified amino acids, the amine or acidic function of the amino acid has reacted with a chemical reagent. This is often done to protect these chemical amine and acid functions of the amino acid in a subsequent reaction or to give special properties to the amino acids, like improved solubility. Examples of such chemical modifications are benzyloxycarbonyl, aminobutyric acid, butyl ester, pyroglutamic acid. More examples of common modifications of amino acids and small amino acid fragments can be found in the Bachem, 1996, Peptides and Biochemicals Catalog.

Preferred polyamino acids are polylysines, polyarginine, polyglutamine, polyasparagine, polyhistidine, polytryptophane or mixtures thereof. Most preferred are polylysines or polyamino acids where more than 50% of the amino acids are lysine, since the primary amine function in the side chain of the lysine is the most reactive amine of all amino acids.

The preferred polyamino acid has a molecular weight of 500 to 10.000.000, more preferably between 2.000 and 25.000.

The polyamino acid can be cross linked. The cross linking can be obtained for example by condensation of the amine group in the side chain of the amino acid like lysine with the carboxyl function on the amino acid or with protein cross linkers like PEG derivatives. The cross linked polyamino acids still need to have free primary and/or secondary amino groups left for reaction with the active ingredient.

The preferred cross linked polyamino acid has a molecular weight of 20.000 to 10.000.000, more preferably between 200.000 and 2.000.000.

The polyamino acid or the amino acid can be co-polymerized with other reagents like for instance with acids, amides, acyl chlorides. More specifically with aminocaproic acid, adipic acid, ethylhexanoic acid, caprolactam or mixture thereof. The molar ratio used in these copolymers ranges from 1:1 (reagent/amino acid (lysine)) to 1:20, more preferably from 1:1 to 1:10.

The polyamino acid like polylysine can also be partially ethoxylated.

Examples and supply of polyaminoacids containing lysine, arginine, glutamine, asparagine are given in the Bachem 1996, Peptides and Biochemicals catalog.

The polyaminoacid can be obtained before reaction with the active ingredient, under a salt form. For example polylysine can be supplied as polylysine hydrobromide. Polylysine hydrobromide is commercially available from Sigma, Applichem, Bachem and Fluka.

Examples of suitable amino functional polymers containing at least one primary and/or secondary amine group for the purpose of the present invention are:

Polyvinylamine with a MW of about 300-2.10E6;

Polyvinylamine alkoxylated with a MW of about 600, 1200 or 3000 and an ethoxylation degree of 0.5;

Polyvinylamine vinylalcohol—molar ratio 2:1, polyvinylaminevinylformamide—molar ratio 1:2 and polyvinylamine vinylformamide-molar ratio 2:1;

Triethylenetetramine, diethylenetriamine, tetraethylenepentamine;

Bis-aminopropylpiperazine;

Polyamino acid (L-lysine/lauric acid in a molar ratio of 10/1), Polyamino acid (L-lysine/aminocaproic acid/adipic acid in a molar ratio of 5/5/1), ), Polyamino acid (L-lysine/aminocaproic acid/ethylhexanoic acid in a molar ratio of 5/3/1) Polyamino acid (polylysine-cocaprolactam); Polylysine; Polylysine hydrobromide; cross-linked polylysine,

amino substituted polyvinylalcohol with a MW ranging from 400-300,000;

polyoxyethylene bis [amine] available from e.g. Sigma;

polyoxyethylene bis [6-aminohexyl] available from e.g. Sigma;

N,N′-bis-(3-aminopropyl)-1,3-propanediamine linear or branched (TPTA); and

1,4-bis-(3-aminopropyl) piperazine (BNPP).

The more preferred compounds are selected from ethyl-4-amino benzoate, polyethyleneimine polymers commercially available under the tradename Lupasol like Lupasol HF, P, PS, SK, SNA; the diaminobutane dendrimers Astramol®, polylysine, cross-linked polylysine, N,N′-bis-(3-aminopropyl)-1,3-propanediamine linear or branched; 1,4-bis-(3-aminopropyl) piperazine, and mixtures thereof. Even most preferred compounds are those selected from ethyl-4-amino benzoate, polyethyleneimine polymers commercially available under the tradename Lupasol like Lupasol HF, P, PS, SK, SNA; polylysine, cross-linked polylysine, N,N′-bis-(3-aminopropyl)-1,3-propanediamine linear or branched, 1,4-bis-(3-aminopropyl) piperazine, and mixtures thereof.

Advantageously, such most preferred primary and/or secondary amine compounds also provide fabric appearance benefit, in particular colour appearance benefit, thus providing a resulting amine reaction product with the properties of fabric appearance benefit, deposition onto the surface to be treated, and delayed release of the active as well as release of the perfume composition. Further, when the primary and/or secondary amine compound has more than one free primary and/or secondary amine group, several different active ingredients (aldehyde and/or ketone) can be linked to the amine compound.

Of course, the primary and/or secondary amine compound may also be used as is, i.e. without having been reacted with the above benefit agent like aldehyde and/or ketone perfume ingredient, but with a benefit agent like a perfume composition which is entrapped or embedded within the primary and/or secondary amine compound. Moreover, the primary and/or secondary amine compound may also be reacted with compounds other than the benefit agent mentioned above like acyl halides, like acetylchloride, palmytoyl chloride or myristoyl chloride, acid anhydrides like acetic anhydride, alkylhalides or arylhalides to do alkylation or arylation, aldehydes or ketones not used as perfume ingredients like formaldehyde, glutaraldehyde, unsaturated ketones, aldehydes or carboxylic acids like 2-decylpropenoic acid, propenal, propenone to form reaction products with the required viscosity.

B-Benefit Agent

The benefit agent is a component that will react with the amino-functional polymer and provide a beneficial effect on the treated surface like fabric upon contact with water or humidity. Hence, the benefit agent may be selected from a flavour ingredient, a pharmaceutical ingredient, a biocontrol ingredient, a perfume composition, a refreshing cooling ingredient and mixtures thereof.

Typically, the benefit agent comprises from 10 to 90%, preferably from 30 to 85%, more preferably from 45 to 80% by weight of the pro-perfume component.

Flavour ingredients include spices, flavor enhancers that contribute to the overall flavour perception.

Pharmaceutical ingredients include drugs.

Biocontrol ingredients include biocides, antimicrobials, bactericides, fungicides, algaecides, mildewcides, disinfectants, antiseptics, insecticides, vermicides, plant growth hormones.

Typical antimicrobials which can be carried by the carrier material include amine oxide surfactants, photo-activated bleaches, chlorhexidine diacetate, glutaraldehyde, cinnamon oil and cinnamaldehyde, citric acid, decanoic acid, lactic acid, maleic acid, nonanoic acid, polybiguanide, propylene glycol, cumene sulfonate, eugenol, thymol, benzalkonium chloride, geraniol, and mixtures thereof. Preferred are compounds which can react with the carrier material. Preferably, the carrier material is a polymer, preferably a polymer which is reacted with another benefit agent, such as for example perfumes described herein, and this polymer or polymer reaction product functions as a carrier for the biocide. Preferred carried compositions for use in fabric care and cleaning compositions have a viscosity of at least 500,000 or even at least 1,000,000 cps, as described hereinafter. Preferred polymers are also described in more detail hereinafter.

Typical insect and/or moth repellants are perfume ingredients, such as citronellal, citral, N, N diethyl meta toluamide, Rotundial, 8-acetoxycarvotanacetone, and mixtures thereof. Other examples of insect and/or moth repellant for use herein are disclosed in U.S. Pat. Nos. 4,449,987, 4,693,890, 4,696,676, 4,933,371, 5,030,660, 5,196,200, and “Semio Activity of Flavor and Fragrance molecules on various Insect Species”, B. D. Mookherjee et al., published in Bioactive Volatile Compounds from Plants, ASC Symposium Series 525, R. Teranishi, R. G. Buttery, and H. Sugisawa, 1993, pp. 35-48.

One preferred benefit agent is a perfume ingredient. One typical perfume ingredient is a aldehyde perfume ingredient. Preferably, the perfume aldehyde is selected from adoxal; anisic aldehyde; cymal; ethyl vanillin; florhydral; helional; heliotropin; hydroxycitronellal; koavone; lauric aldehyde; lyral; methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)propanal, 2-methyl-3-(para-methoxyphenyl propanal, 2-methyl-4-(2,6,6-trimethyl-2(1)-cyclohexen-1-yl) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro4,7-methano-1H-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carboxaldehyde, 5 or 6 methoxy0hexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, para-tolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro4,7-methanoindan-1-carboxaldehyde, 2-methyl octanal, alpha-methyl-4-(1-methyl ethyl) benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, 1-p-menthene-q-carboxaldehyde, citral, lilial and mixtures thereof.

More preferred aldehydes are selected from citral, 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P.T. Bucinal, lyral, cymal, methyl nonyl acetaldehyde, trans-2-nonenal, lilial, trans-2-nonenal, and mixture thereof.

Another typical perfume ingredient is a ketone perfume ingredient. Preferably, the perfume ketone is selected from buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-Ionone, Beta-Ionone, Gamma-Methyl so-called Ionone, Fleuramone, Dihydrojasmone, Cis-Jasmone, Iso-E-Super, Methyl-Cedrenyl-ketone or Methyl-Cedrylone, Acetophenone, Methyl-Acetophenone, Para-Methoxy-Acetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethyl-Cyclohexanone, Methyl-Heptenone, 2-(2-(4-Methyl-3-cyclohexen-1-yl)propyl)-cyclopentanone, 1-(p-Menthen-6(2)-yl)-1-propanone, 4-(4-Hydroxy-3-methoxyphenyl)-2-butanone, 2-Acetyl-3,3-Dimethyl-Norbornane, 6,7-Dihydro-1,1,2,3,3-Pentamethyl-4(5H)-Indanone, 4-Damascol, Dulcinyl or Cassione, Gelsone, Hexalon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, Tetrameran, hedione, and mixtures thereof.

More preferably, for the above mentioned compounds, the preferred ketones are selected from Alpha Damascone, Delta Damascone, Iso Damascone, Carvone, Gamma-Methyl-Ionone, Iso-E-Super, 2,4,4,7-Tetramethyl-oct-6-en-3-one, Benzyl Acetone, Beta Damascone, Damascenone, methyl dihydrojasmonate, methyl cedrylone, hedione, and mixtures thereof.

Still, the benefit agent may also comprise a perfume composition made of mixture of perfume ingredients including or not the above mentioned aldehyde or ketone. This composition is then entrapped within the pro-perfume component by mixing. By such means, a more fully complete perfume formulation can then be deposited onto the contacted surface.

Typical of these ingredients include fragrant substance or mixture of substances including natural (i.e., obtained by extraction of flowers, herbs, leaves, roots, barks, wood, blossoms or plants), artificial (i.e., a mixture of different nature oils or oil constituents) and synthetic (i.e., synthetically produced) odoriferous substances. Such materials are often accompanied by auxiliary materials, such as fixatives, extenders, stabilizers and solvents. These auxiliaries are also included within the meaning of “perfume”, as used herein. Typically, perfumes are complex mixtures of a plurality of organic compounds.

Suitable perfumes are disclosed in U.S. Pat. No. 5,500,138, said patent being incorporated herein by reference.

Examples of perfume ingredients useful in the perfume compositions include, but are not limited to, amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma. Additional examples of fragrance materials include, but are not limited to, orange oil; lemon oil; grapefruit oil; bergamot oil; clove oil; dodecalactone gamma; methyl-2-(2-pentyl-3-oxo-cyclopentyl) acetate; beta-naphthol methylether; methyl-beta-naphthylketone; coumarin; 4-tert-butylcyclohexyl acetate; alpha,alpha-dimethylphenethyl acetate; methylphenylcarbinyl acetate; cyclic ethyleneglycol diester of tridecandioic acid; 3,7-dimethyl-2,6-octadiene-1-nitrile; ionone gamma methyl; ionone alpha; ionone beta; petitgrain; methyl cedrylone; 7-acetyl-1,2,3,4,5,6,7,8-octahydro-1,1,6,7-tetramethyl-naphthalene; ionone methyl; methyl-1,6,10-trimethyl-2,5,9-cyclododecatrien-1-yl ketone; 7-acetyl-1,1,3,4,4,6-hexamethyl tetralin; 4-acetyl-6-tert-butyl-1,1-dimethyl indane; benzophenone; 6-acetyl-1,1,2,3,3,5-hexamethyl indane; 5-acetyl-3-isopropyl-1,1,2,6-tetramethyl indane; 1-dodecanal; 7-hydroxy-3,7-dimethyl octanal; 10-undecen-1-al; iso-hexenyl cyclohexyl carboxaldehyde; formyl tricyclodecan; cyclopentadecanolide; 16-hydroxy-9-hexadecenoic acid lactone; 1,3,4,6,7,8-hexahydro-4,6,6,7,8,8-hexamethylcyclopenta-gamma-2-benzopyrane; ambroxane; dodecahydro-3a,6,6,9a-tetramethylnaphtho-[2,1b]furan; cedrol; 5-(2,2,3-trimethylcyclopent-3-enyl)-3-methylpentan-2-ol; 2-ethyl-4-(2,2,3-trimethyl-3-cyclopenten-1-yl)-2-buten-1-ol; caryophyllene alcohol; cedryl acetate; para-tert-butylcyclohexyl acetate; patchouli; olibanum resinoid; labdanum; vetivert; copaiba balsam; fir balsam; hydroxycitronellal and indol; phenyl acetaldehyde and indol;

More examples of perfume components are geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methylether; isolongifolanone; aubepine nitrile; aubepine; heliotropine; eugenol; vanillin; diphenyl oxide; hydroxycitronellal ionones; methyl ionones; isomethyl ionomes; irones; cis-3-hexenol and esters thereof; indane musk fragrances; tetralin musk fragrances; isochroman musk fragrances; macrocyclic ketones; macrolactone musk fragrances; ethylene brassylate. Also suitable herein as perfume ingredients of the perfume composition are the so-called Schiff base. Schiff-Bases are the condensation of an aldehyde perfume ingredient with an anthranilate. A typical description can be found in U.S. Pat. No. 4,853,369. Typical of Schiff bases are selected from Schiff's base of 4-(4-hydroxy-4-methylpentyl)-3-cyclohexene-1-carboxaldehyde and methyl anthranilate; condensation products of: hydroxycitronellal and methyl anthranilate; 4-(4-hydroxy-4-methyl pentyl)-3-cyclohexene-1-carboxaldehyde and methyl anthranilate; Methyl Anthranilate and HydroxyCitronellal commercially available under the tradename Aurantiol; Methyl Anthranilate and Methyl Nonyl Acetaldehyde commercially available under the tradename Agrumea; Methyl Anthranilate and PT Bucinal commercially available under the tradename Verdantiol; Methyl anthranilate and Lyral commercially available under the tradename Lyrame; Methyl Anthranilate and Ligustral commercially available under the tradename Ligantral; and mixtures thereof.

Preferably, the perfume ingredients and/or compositions useful in the present invention compositions are substantially free of halogenated materials and nitromusks.

More preferably, the perfume compounds are characterised by having a low Odor Detection Threshold. Such Odor Detection Threshold (ODT) should be lower than 1 ppm, preferably lower than 10 ppb—measured at controlled Gas Chromatography (GC) conditions such as described here below. This parameter refers to the value commonly used in the perfumery arts and which is the lowest concentration at which significant detection takes place that some odorous material is present. Please refer for example in “Compilation of Odor and Taste Threshold Value Data (ASTM DS 48 A)”, edited by F. A. Fazzalari, International Business Machines, Hopwell Junction, N.Y. and in Calkin et al., Perfumery, Practice and Principles, John Willey & Sons, Inc., page 243 et seq (1994). For the purpose of the present invention, the Odor Detection Threshold is measured according to the following method: The gas chromatograph is characterized to determine the exact volume of material injected by the syringe, the precise split ratio, and the hydrocarbon response using a hydrocarbon standard of known concentration and chain-length distribution. The air flow rate is accurately measured and, assuming the duration of a human inhalation to last 0.02 minutes, the sampled volume is calculated. Since the precise concentration at the detector at any point in time is known, the mass per volume inhaled is known and hence the concentration of material. To determine the ODT of a perfume material, solutions are delivered to the sniff port at the back-calculated concentration. A panelist sniffs the GC effluent and identifies the retention time when odor is noticed. The average over all panelists determines the threshold of noticeability. The necessary amount of analyte is injected onto the column to achieve a certain concentration, such as 10 ppb, at the detector. Typical gas chromatograph parameters for determining odor detection thresholds are listed below.

GC: 5890 Series II with FID detector

7673 Autosampler

Column: J&W Scientific DB-1

Length 30 meters ID 0.25 mm film thickness 1 micron

Method:

Split Injection: 17/1 split ratio

Autosampler: 1.13 microliters per injection

Column Flow: 1.10 mL/minute

Air Flow: 345 mL/minute

Inlet Temp. 245° C.

Detector Temp. 285° C.

Temperature Information

Initial Temperature: 50° C.

Rate: 5C./minute

Final Temperature: 280° C.

Final Time: 6 minutes

Leading assumptions: 0.02 minutes per sniff

GC air adds to sample dilution

Examples of such preferred perfume components are those selected from : 2-methyl-2-(para-iso-propylphenyl)-propionaldehyde, 1-(2,6,6-trimethyl-2-cyclohexan-1-yl)-2-buten-1-one and/or para-methoxy-acetophenone. Even more preferred are the following compounds having an ODT£ 10 ppb measured with the method described above: undecylenic aldehyde, undecalactone gamma, heliotropin, dodecalactone gamma, p-anisic aldehyde, para hydroxy-phenyl-butanone, cymal, benzyl acetone, ionone alpha, p.t.bucinal, damascenone, ionone beta, methyl-nonyl ketone, methyl heptine carbonate, linalool, indol, cis-3-hexenyl salicylate, vanillin, methyl isobutenyl tetrahydropyran, ethylvanillin, coumarin, ethyl methyl phenyl glycidate, eugenol, methylanthranilate, iso eugenol, beta naphtol methyl ester, herbavert, lyral, allyl amyl glycolate, dihydro iso jasmonate, ethyl-2-methylbutyrate, nerol, and phenylacetaldehyde. Most preferably the perfume composition comprises at least 5%, more preferably at least 10% of such components

Most preferably, the perfume ingredients are those as described in WO 96/12785 on page 12-14. Even most preferred are those perfume compositions comprising at least 10%, preferably 25%, by weight of perfume ingredient with an ClogP of at least 2.0, preferably of at least 3.0 and boiling point of at least 250C. Still another preferred perfume composition is a composition comprising at least 20%, preferably 35%, by weight of perfume ingredient with an ClogP at least 2.0, more preferably of at least 3.0 and boiling point of less than or equal to 250C. Clog P is a commonly known calculated measure as defined in the following references “Calculating log P_{oct} from Structures”; Albert Leo (Medicinal Chemistry Project, Pomona College, Claremont, Calif. USA. Chemical Reviews, Vol. 93, number 4, June 1993; as well as from Comprehensive Medicinal Chemistry, Albert Leo, C. Hansch, Ed. Pergamon Press: Oxford, 1990, Vol. 4, p.315; and Calculation Procedures for molecular lipophilicity: a comparative Study, Quant. Struct. Act. Realt. 15, 403-409 (1996), Raymund Mannhold and Karl Dross.

Typically, the level of pro-perfume component is of from 10 to 70% by weight, preferably of from 40 to 60% by weight, most preferably of 50% by weight of the pro-perfume composition.

Suspending Material

A suspending material is another essential element of the present invention. Indeed, a suspending material will provide improved incorporation and stability within the detergent composition, especially liquid detergent compositions.

In addition, when incorporated into aqueous liquid detergent composition, the suspending material will preferably protect the pro-perfume component from the aqueous medium.

Typically, the level of suspending aid is of from 30 to 90% by weight, preferably of from 45 to 75% by weight, most preferably of from 50% to 70% by weight of the pro-perfume composition.

Typical of such suspending materials includes liquid as well as solid suspending aid. The liquid suspending material is preferably in a hydrophobic form.

As used herein in relation to suspending materials, “hydrophobic” means substantially water insoluble. In this regard, “substantially water insoluble” shall refer to a material that is not soluble in distilled (or equivalent) water, at 25° C., at a concentration of 0.2% by weight, and preferably not soluble at 0.1% by weight (calculated on a water plus suspending aid weight basis). The terms “soluble”, “solubility” and the like, for purposes hereof, corresponds to the maximum concentration of suspending material, as applicable, that can dissolve in water or other solvents to form a homogeneous solution, as is well understood to those skilled in the art.

Typical of materials that are in hydrophobic form and are useful as suspending materials include ingredients useful in perfumery. Typical of such ingredients are the perfume raw materials, solvents, and mixtures thereof. Typical hydrophobic raw materials include the ketones or aldehydes like hexyl cinnamic aldehyde, hydrocarbons like Limolene, d-Limonene, esters like Hercolyn D, benzyl salicylate, hexyl salicylate, triethyl citrate, iso propyl myristate, or mixtures thereof. Typical hydrophobic solvents include diethyl phtalate, ethers like butoxypropoxypropanol, dipropylene glycol, and mixtures thereof.

Still other suitable suspending materials are the silicone materials. The word “silicone” as used herein includes emulsified and/or microemulsified silicones, including those that are commercially available and those that are emulsified and/or microemulsified in the composition.

Some non-limiting examples of silicones which are useful in the present invention are: cyclic silicones; non-volatile silicone fluids such as polydimethyl siloxane gums and fluids; such as linear silicone polymer fluids having the formula (CH₃)3SiO[(CH3)2SiO]mSi(CH)3 where m is 0 or more and whereby m has an average value such that the viscosity at 25° C. of the silicone fluid is preferably 5 centistokes or more, more preferably 500 centistokes or more, the silicone fluid preferably having a weight average molecular weight of 800 or more, preferably 25,000 or more; or such as volatile silicone fluid which can be a cyclic silicone fluid of the formula [(CH3)2SiO]n where n ranges between about 3 to about 7, preferably about 5 or 6; or such as silicone surfactants, such as polyglycolethers; other suitable silicone surfactants are desribed in ‘Silicone Surfactants’, by R. M. Hill, ISBN 0-8247-0010-4, 1999, Marcel Dekker Inc. New York, Basel. These silicone surfactants can be ABA type copolymers, grafted copolymers or ter- or tri-siloxane polymers. The silicone surfactants can be silicone polyether copolymers and can have ethylene oxide, propylene oxide or butylene oxide based chains and/or mixtures thereof. More preferably the silicone surfactant has a weight average molecular wiegth of more than 1000, more preferably more than 2500 or even more than 3500 or even 5000 or more. The silicone or silicone surfactants can be a fluorosilicone as well, preferably a fluorosilicone with a viscosity of at least 1000 centistokes.

The silicones and fluorosilicones also preferably have a weight average molecular weight of 1000 or more, preferaby 2500 or more or even 3500 or more, or even 500 or more.

Preferred may also be that the silicone surfactant has a HLB (-EO) value 5 or less, or even 3 or less, as can be determined by known methods.

Suitable examples of silicone for use herein include silicones commercially available from Dow Corning Corporation like the DC 3225C; DC5225C and DC246 for cyclic silicones; silicone glycols ethers like DC5200, DCC1248, DC190; the DC 244 Fluids, DC 245 Fluids, DC 344 Fluids, or DC 345 Fluids, or ABIL K4, ABIL B 8839 for the cyclomethicone, or the DC 200 fluids, ABIL K 520 (hexamethyl disiloxane), ABIL 10 to ABIL 100000 (dimethicone), ABIL AV 8853 (Phenyl dimethicone) for the linear silicones; Dow Corning's FS1265 fluorosilicone.

Highly preferred are DC 3225C, DC5225 and dimethylpolysiloxanes available from Sigma and Dow Corning.

Still another suitable method is to use a combination of various silicone materials, such as those described herein before. Preferably, one or more silicone material(s) is (are) emulsified or microemulsified in one or more other silicone materails, forming thus the suspending material herein. The pro-perfume component can then be suspended herein.

Preferably, at least 80% or even 90% of such a silicone mixture is formed by 2 silicone materials. Then, the weight ratio of a first silicone material to a second silicone material in such a mixture is preferably from 1:50 to 2:1, more preferably 1:19 to 3:2, or even 1:9 to 1:1.

Furthermore, particles with a mean particle size up to 20 microns or even up to 10 microns, preferably 1 to 10 microns, can be added to the silicone mixture, like silica, quarz, TiO₂, for density adjustment of the suspensing material, or pro-perfume composition.

Preferably, for the purpose of the invention, the suspending material has a viscosity between 250 and 250000 cps, more preferably between 700 and 70000 cps. More preferably, the suspending material is selected from benzyl salicylate, diethyl phatalate, dipropylene glycol, butoxypropoxypropanol, silicones, and mixtures thereof, most preferably is selected from silicones.

Method for Making the Pro-Perfume Composition

Typically, the close physical proximity between the pro-perfume and the suspending aid is obtained by mixing together the reacted amino-functional component and the suspending aid, typically present in a 50:50 weight ratio, under high shear mixing (i.e. 50.000 rpm) using an Ultra Turrax apparatus at a temperature of 50-60° C. for 5 minutes.

Particle Size

For ease of handling and incorporation, e.g. suspension into the detergent compositions of the invention, it is preferred to provide droplets of specified size. Typically, this is obtained upon high shear mixing of the above mentioned mixture. However, this can also be obtained by means of a twin screw extruder which provide one convenient way for making the pro-perfume composition in industrial quantities via a continous process like by means of a twin Screw Extruder (TSE). Suitable TSE include the TX-57 MAG, TX-85 MAG, TX-110 MAG, TX-144 MAG, or TX-178 MAG twin screw extruder from Wenger. One preferred for use herein is the TX-57 MAG. TSE suitable for use herein comprise at one of their extremities so called herein after “first part of the TSE” two distinct inlet: one for the active and the other for the amine, and at about the middle of the TSE, so called hereinafter “second part of the TSE” another inlet for the suspending aid. Temperature controllers are also distributed along the TSE.

Typically, the particle size of the droplet within the detergent composition is of from 1 microns to 50 microns, more preferably from 2 or evne from 5 micron to 30 microns, and most preferably up to 20 or even up to 15 microns.

Incorporation into Detergent Composition

The pro-perfume composition is then incorporated in a detergent composition. Means of incorporation into the detergent composition are conventionally known in the art, and is typically made depending on its end form by either spraying when in sprayable liquid form, or dry-addition, or mixing when in liquid viscous form, e.g. having a viscosity between 100 and 1000 cps. The viscosity is measured on a rheometer, TA Instrument CSL²₁₀₀ at a temperature of 20° C. with a gap setting of 500 microns.

Preferably, the pro-perfume composition is incorporated by mixing.

Typical level of incorporation of the pro-perfume composition within the detergent composition are of from 0.01 to 10%, more preferably from 0.02 to 5%, and most preferably from 0.03, or even 0.1 or even 0.2 to 1% by weight of the detergent composition.

The detergent composition typically comprises one or more detergent ingredient. Preferably, the detergent composition is a liquid detergent composition, including aqueous as well as non-aqueous liquid detergent compositions.

The aqueous and non-aqueous liquid detergent compositions of this invention typically comprises one or more detergent ingredient. Suitable detergent ingredients comprise a surfactant system.

### Non-Aqueous Baded Heavy Duty Liquid Detergents Surfactant-Containing Liquid Phase

The present invention liquid detergent composition comprises non-aqueous, liquid, heavy-duty detergent compositions in the form of a stable suspension of solid, substantially insoluble particulate material dispersed throughout a structured, surfactant-containing liquid phase. The detergent composition comprises from about 49% to 99.95% by weight of the composition of a structured, surfactant-containing liquid phase formed by combining:

i) from about 1% to 80% by weight of said liquid phase of one or more nonaqueous organic diluents; and

ii) from about 20% to 99% by weight of said liquid phase of a surfactant system comprising surfactants selected from the group consisting of anionic, nonionic, cationic surfactants and combinations thereof.

The surfactant-containing, non-aqueous liquid phase of the present invention will generally comprise from about 52% to about 98.9% by weight of the detergent compositions herein. More preferably, this liquid phase is surfactant-structured and will comprise from about 55% to 98% by weight of the compositions. Most preferably, this non-aqueous liquid phase will comprise from about 55% to 70% by weight of the compositions herein. Such a surfactant-containing liquid phase will frequently have a density of from about 0.6 to 1.4 g/cc, more preferably from about 0.9 to 1.3 g/cc. The liquid phase of the detergent compositions herein is preferably formed from one or more non-aqueous organic diluents into which is mixed a surfactant structuring agent which is preferably a specific type of anionic surfactant-containing powder.

Non-Aqueous Organic Diluents

The major component of the liquid phase of the detergent compositions herein comprises one or more non-aqueous organic diluents. The non-aqueous organic diluents used in this invention may be either surface active, i.e., surfactant, liquids or non-aqueous, non-surfactant liquids referred to herein as non-aqueous solvents. The term “solvent” is used herein to connote the non-surfactant, non-aqueous liquid portion of the compositions herein. While some of the essential and/or optional components of the compositions herein may actually dissolve in the “solvent”-containing liquid phase, other components will be present as particulate material dispersed within the “solvent”-containing liquid phase. Thus the term “solvent” is not meant to require that the solvent material be capable of actually dissolving all of the detergent composition components added thereto.

The non-aqueous liquid diluent component will generally comprise from about 50% to 100%, more preferably from about 50% to 80%, most preferably from about 55% to 75%, of a structured, surfactant-containing liquid phase. Preferably the liquid phase of the compositions herein, i.e., the non-aqueous liquid diluent component, will comprise both non-aqueous liquid surfactants and non-surfactant non-aqueous solvents.

i) Non-Aqueous Surfactant Liquids

Suitable types of non-aqueous surfactant liquids which can be used to form the liquid phase of the compositions herein include the alkoxylated alcohols, ethylene oxide (EO)-propylene oxide (PO) block polymers, polyhydroxy fatty acid amides, alkylpolysaccharides, and the like. Such normally liquid surfactants are those having an HLB ranging from 10 to 16. Most preferred of the surfactant liquids are the alcohol alkoxylate nonionic surfactants.

Alcohol alkoxylates are materials which correspond to the general formula:

R¹ (CₘH₂ₘO)ₙOH

wherein R¹ is a C₈-C₁₆ alkyl group, m is from 2 to 4, and n ranges from about 2 to 12. Preferably R¹ is an alkyl group, which may be primary or secondary, that contains from about 9 to 15 carbon atoms, more preferably from about 10 to 14 carbon atoms. Preferably also the alkoxylated fatty alcohols will be ethoxylated materials that contain from about 2 to 12 ethylene oxide moieties per molecule, more preferably from about 3 to 10 ethylene oxide moieties per molecule.

The alkoxylated fatty alcohol materials useful in the liquid phase will frequently have a hydrophilic-lipophilic balance (HLB) which ranges from about 3 to 17. More preferably, the HLB of this material will range from about 6 to 15, most preferably from about 8 to 15.

Examples of fatty alcohol alkoxylates useful in or as the non-aqueous liquid phase of the compositions herein will include those which are made from alcohols of 12 to 15 carbon atoms and which contain about 7 moles of ethylene oxide. Such materials have been commercially marketed under the trade names Neodol 25-7 and Neodol 23-6.5 by Shell Chemical Company. Other useful Neodols include Neodol 1-5, an ethoxylated fatty alcohol averaging 11 carbon atoms in its alkyl chain with about 5 moles of ethylene oxide; Neodol 23-9, an ethoxylated primary C₁₂-C₁₃ alcohol having about 9 moles of ethylene oxide and Neodol 91-10, an ethoxylated C₉-C₁₁ primary alcohol having about 10 moles of ethylene oxide. Alcohol ethoxylates of this type have also been marketed by Shell Chemical Company under the Dobanol tradename. Dobanol 91-5 is an ethoxylated C₉-C₁₁ fatty alcohol with an average of 5 moles ethylene oxide and Dobanol 25-7 is an ethoxylated C₁₂-C₁₅ fatty alcohol with an average of 7 moles of ethylene oxide per mole of fatty alcohol.

Other examples of suitable ethoxylated alcohols include Tergitol 15-S-7 and Tergitol 15-S-9 both of which are linear secondary alcohol ethoxylates that have been commercially marketed by Union Carbide Corporation. The former is a mixed ethoxylation product of C₁₁ to C₁₅ linear secondary alkanol with 7 moles of ethylene oxide and the latter is a similar product but with 9 moles of ethylene oxide being reacted.

Other types of alcohol ethoxylates useful in the present compositions are higher molecular weight nonionics, such as Neodol 45-11, which are similar ethylene oxide condensation products of higher fatty alcohols, with the higher fatty alcohol being of 14-15 carbon atoms and the number of ethylene oxide groups per mole being about 11. Such products have also been commercially marketed by Shell Chemical Company.

If alcohol alkoxylate nonionic surfactant is utilized as part of the non-aqueous liquid phase in the detergent compositions herein, it will preferably be present to the extent of from about 1% to 60% of the composition structured liquid phase. More preferably, the alcohol alkoxylate component will comprise about 5% to 40% of the structured liquid phase. Most preferably, an alcohol alkoxylate component will comprise from about 5% to 35% of the detergent composition structured liquid phase. Utilization of alcohol alkoxylate in these concentrations in the liquid phase corresponds to an alcohol alkoxylate concentration in the total composition of from about 1% to 60% by weight, more preferably from about 2% to 40% by weight, and most preferably from about 5% to 25% by weight, of the composition.

Another type of non-aqueous surfactant liquid which may be utilized in this invention are the ethylene oxide (EO)-propylene oxide (PO) block polymers. Materials of this type are well known nonionic surfactants which have been marketed under the tradename Pluronic. These materials are formed by adding blocks of ethylene oxide moieties to the ends of polypropylene glycol chains to adjust the surface active properties of the resulting block polymers. EO-PO block polymer nonionics of this type are described in greater detail in Davidsohn and Milwidsky; Synthetic Detergents, 7th Ed.; Longman Scientific and Technical (1987) at pp. 34-36 and pp. 189-191 and in U.S. Pat. No. 2,674,619 and 2,677,700. All of these publications are incorporated herein by reference. These Pluronic type nonionic surfactants are also believed to function as effective suspending agents for the particulate material which is dispersed in the liquid phase of the detergent compositions herein.

Another possible type of non-aqueous surfactant liquid useful in the compositions herein comprises polyhydroxy fatty acid amide surfactants. If present, the polyhydroxy fatty acid amide surfactants are preferably present in a concentration of from about 0.1 to about 8%. Materials of this type of nonionic surfactant are those which conform to the formula:

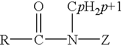

wherein R is a C₉₋₁₇ alkyl or alkenyl, p is from 1 to 6, and Z is glycityl derived from a reduced sugar or alkoxylated derivative thereof. Such materials include the C₁₂-C₁₈ N-methyl glucamides. Examples are N-methyl N-1-deoxyglucityl cocoamide and N-methyl N-1-deoxyglucityl oleamide. Processes for making polyhydroxy fatty acid, amides are know and can be found, for example, in Wilson, U.S. Pat. No. 2,965,576 and Schwartz, U.S. Pat. No. 2,703,798, the disclosures of which are incorporated herein by reference. The materials themselves and their preparation are also described in greater detail in Honsa, U.S. Pat. No. 5,174,937, Issued Dec. 26, 1992, which patent is also incorporated herein by reference.

The amount of total liquid surfactant in the preferred surfactant-structured, non-aqueous liquid phase herein will be determined by the type and amounts of other composition components and by the desired composition properties. Generally, the liquid surfactant can comprise from about 35% to 70% of the non-aqueous liquid phase of the compositions herein. More preferably, the liquid surfactant will comprise from about 50% to 65% of a non-aqueous structured liquid phase. This corresponds to a non-aqueous liquid surfactant concentration in the total composition of from about 15% to 70% by weight, more preferably from about 20% to 50% by weight, of the composition.

ii) Non-Surfactant Non-Aqueous Organic Solvents

The liquid phase of the detergent compositions herein may also comprise one or more non-surfactant, non-aqueous organic solvents. Such non-surfactant non-aqueous liquids are preferably those of low polarity. For purposes of this invention, “low-polarity” liquids are those which have little, if any, tendency to dissolve one of the preferred types of particulate material used in the compositions herein, i.e., the peroxygen bleaching agents, sodium perborate or sodium percarbonate. Thus relatively polar solvents such as ethanol are preferably not utilized. Suitable types of low-polarity solvents useful in the non-aqueous liquid detergent compositions herein do include non-vicinal C₄-C₈ alkylene glycols, alkylene glycol mono lower alkyl ethers, lower molecular weight polyethylene glycols, lower molecular weight methyl esters and amides, and the like.

A preferred type of non-aqueous, low-polarity solvent for use in the compositions herein comprises the non-vicinal C₄-C₈ branched or straight chain alkylene-glycols. Materials of this type include hexylene glycol (4-methyl-2,4-pentanediol), 1,6-hexanediol, 1,3-butylene glycol and 1,4-butylene glycol. Hexylene glycol is the most preferred.

Another preferred type of non-aqueous, low-polarity solvent for use herein comprises the mono-, di-, tri-, or tetra-C₂-C₃ alkylene glycol mono C₂-C₆ alkyl ethers. The specific examples of such compounds include diethylene glycol monobutyl ether, tetraethylene glycol monobutyl ether, dipropolyene glycol monoethyl ether, and dipropylene glycol monobutyl ether. Diethylene glycol monobutyl ether, dipropylene glycol monobutyl ether and butoxy-propoxy-propanol (BPP) are especially preferred. Compounds of the type have been commercially marketed under the tradenames Dowanol, Carbitol, and Cellosolve.

Another preferred type of non-aqueous, low-polarity organic solvent useful herein comprises the lower molecular weight polyethylene glycols (PEGs). Such materials are those having molecular weights of at least about 150. PEGs of molecular weight ranging from about 200 to 600 are most preferred.

Yet another preferred type of non-polar, non-aqueous solvent comprises lower molecular weight methyl esters. Such materials are those of the general formula: R¹—C(O)—OCH₃ wherein R¹ ranges from 1 to about 18. Examples of suitable lower molecular weight methyl esters include methyl acetate, methyl propionate, methyl octanoate, and methyl dodecanoate.

The non-aqueous, generally low-polarity, non-surfactant organic solvent(s) employed should, of course, be compatible and non-reactive with other composition components, e.g., bleach and/or activators, used in the liquid detergent compositions herein. Such a solvent component is preferably utilized in an amount of from about 1% to 70% by weight of the liquid phase. More preferably, a non-aqueous, low-polarity, non-surfactant solvent will comprise from about 10% to 60% by weight of a structured liquid phase, most preferably from about 20% to 50% by weight, of a structured liquid phase of the composition. Utilization of non-surfactant solvent in these concentrations in the liquid phase corresponds to a non-surfactant solvent concentration in the total composition of from about 1% to 50% by weight, more preferably from about 5% to 40% by weight, and most preferably from about 10% to 30% by weight, of the composition.

iii) Blends of Surfactant and Non-Surfactant Solvents

In systems which employ both non-aqueous surfactant liquids and non-aqueous non-surfactant solvents, the ratio of surfactant to non-surfactant liquids, e.g., the ratio of alcohol alkoxylate to low polarity solvent, within a structured, surfactant-containing liquid phase can be used to vary the rheological properties of the detergent compositions eventually formed. Generally, the weight ratio of surfactant liquid to non-surfactant organic solvent will range about 50:1 to 1:50. More preferably, this ratio will range from about 3:1 to 1:3, most preferably from about 2:1 to 1:2.

Surfactant Structurant

The non-aqueous liquid phase of the detergent compositions of this invention is prepared by combining with the non-aqueous organic liquid diluents hereinbefore described a surfactant which is generally, but not necessarily, selected to add structure to the non-aqueous liquid phase of the detergent compositions herein. Structuring surfactants can be of the anionic, nonionic, cationic, and/or amphoteric types.

Preferred structuring surfactants are the anionic surfactants such as the alkyl sulfates, the alkyl polyalkxylate sulfates and the linear alkyl benzene sulfonates. Another common type of anionic surfactant material which may be optionally added to the detergent compositions herein as structurant comprises carboxylate-type anionics. Carboxylate-type anionics include the C₁₀-C₁₈ alkyl alkoxy carboxylates (especially the EO 1 to 5 ethoxycarboxylates) and the C₁₀-C₁₈ sarcosinates, especially oleoyl sarcosinate. Yet another common type of anionic surfactant material which may be employed as a structurant comprises other sulfonated anionic surfactants such as the C₈-C₁₈ paraffin sulfonates and the C₈-C₁₈ olefin sulfonates. Structuring anionic surfactants will generally comprise from about 1% to 30% by weight of the compositions herein.

As indicated, one preferred type of structuring anionic surfactant comprises primary or secondary alkyl sulfate anionic surfactants. Such surfactants are those produced by the sulfation of higher C₈-C₂₀ fatty alcohols.

Conventional primary alkyl sulfate surfactants have the general formula

ROSO₃—M⁺

wherein R is typically a linear C₈-C₂₀ hydrocarbyl group, which may be straight chain or branched chain, and M is a water-solubilizing cation. Preferably R is a C₁₀₋₁₄ alkyl, and M is alkali metal. Most preferably R is about C₁₂ and M is sodium.

Conventional secondary alkyl sulfates may also be utilized as a structuring anionic surfactant for the liquid phase of the compositions herein. Conventional secondary alkyl sulfate surfactants are those materials which have the sulfate moiety distributed randomly along the hydrocarbyl “backbone” of the molecule. Such materials may be depicted by the structure:

CH₃(CH₂)ₙ(CHOSO₃—M⁺) (CH₂)ₘCH₃

wherein m and n are integers of 2 or greater and the sum of m+n is typically about 9 to 15, and M is a water-solubilizing cation.

If utilized, alkyl sulfates will generally comprise from about 1% to 30% by weight of the composition, more preferably from about 5% to 25% by weight of the composition. Non-aqueous liquid detergent compositions containing alkyl sulfates, peroxygen bleaching agents, and bleach activators are described in greater detail in Kong-Chan et al.; WO 96/10073; Publiched Apr. 4, 1996, which application is incorporated herein by reference.

Another preferred type of anionic surfactant material which may be optionally added to the non-aqueous cleaning compositions herein as a structurant comprises the alkyl polyalkoxylate sulfates. Alkyl polyalkoxylate sulfates are also known as alkoxylated alkyl sulfates or alkyl ether sulfates. Such materials are those which correspond to the formula

R²—O—(CₘH₂ₘO)ₙ—SO₃M

wherein R² is a C10-C22 alkyl group, m is from 2 to 4, n is from about 1 to 15, and M is a salt-forming cation. Preferably, R² is a C12-C18 alkyl, m is 2, n is from about 1 to 10, and M is sodium, potassium, ammonium, alkylammonium or alkanolammonium. Most preferably, R² is a C₁₂-C₁₆, m is 2, n is from about 1 to 6, and M is sodium. Ammonium, alkylammonium and alkanolammonium counterions are preferably avoided when used in the compositions herein because of incompatibility with peroxygen bleaching agents.

If utilized, alkyl polyalkoxylate sulfates can also generally comprise from about 1% to 30% by weight of the composition, more preferably from about 5% to 25% by weight of the composition. Non-aqueous liquid detergent compositions containing alkyl polyalkoxylate sulfates, in combination with polyhydroxy fatty acid amides, are described in greater detail in Boutique et al; PCT Application No. PCT/US96/04223, which application is incorporated herein by reference.

The most preferred type of anionic surfactant for use as a structurant in the compositions herein comprises the linear alkyl benzene sulfonate (LAS) surfactants. In particular, such LAS surfactants can be formulated into a specific type of anionic surfactant-containing powder which is especially useful for incorporation into the non-aqueous liquid detergent compositions of the present invention. Such a powder comprises two distinct phases. One of these phases is insoluble in the non-aqueous organic liquid diluents used in the compositions herein; the other phase is soluble in the non-aqueous organic liquids. It is the insoluble phase of this preferred anionic surfactant-containing powder which can be dispersed in the non-aqueous liquid phase of the preferred compositions herein and which forms a network of aggregated small particles that allows-the final product to stably suspend other solid particulate materials in the composition.

Such a preferred anionic surfactant-containing powder is formed by co-drying an aqueous slurry which essentially contains a) one of more alkali metal salts of C₁₀₋₁₆ linear alkyl benzene sulfonic acids; and b) one or more non-surfactant diluent salts. Such a slurry is dried to a solid material, generally in powder form, which comprises both the soluble and insoluble phases.

The linear alkyl benzene sulfonate (LAS) materials used to form the preferred anionic surfactant-containing powder are well known materials. Such surfactants and their preparation are described for example in U.S. Pat. Nos. 2,220,099 and 2,477,383, incorporated herein by reference. Especially preferred are the sodium and potassium linear straight chain alkylbenzene sulfonates in which the average number of carbon atoms in the alkyl group is from about 11 to 14. Sodium C₁₁₋₁₄, e.g., C₁₂, LAS is especially preferred. The alkyl benzene surfactant anionic surfactants are generally used in the powder-forming slurry in an amount from about 20 to 70% by weight of the slurry, more preferably from about 20% to 60% by weight of the slurry.

The powder-forming slurry also contains a non-surfactant, organic or inorganic salt component that is co-dried with the LAS to form the two-phase anionic surfactant-containing powder. Such salts can be any of the known sodium, potassium or magnesium halides, sulfates, citrates, carbonates, sulfates, borates, succinates, sulfo-succinates and the like. Sodium sulfate, which is generally a bi-product of LAS production, is the preferred non-surfactant diluent salt for use herein. Salts which function as hydrotropes such as sodium sulfo-succinate may also usefully be included. The non-surfactant salts are generally used in the aqueous slurry, along with the LAS, in amounts ranging from about 1 to 50% by weight of the slurry, more preferably from about 5% to 40% by weight of the slurry. Salts that act as hydrotropes can preferably comprise up to about 3% by weight of the slurry.

The aqueous slurry containing the LAS and diluent salt components hereinbefore described can be dried to form the anionic surfactant-containing powder preferably added to the non-aqueous diluents in order to prepare a structured liquid phase within the compositions herein. Any conventional drying technique, e.g., spray drying, drum drying, etc., or combination of drying techniques, may be employed. Drying should take place until the residual water content of the solid material which forms is within the range of from about 0.5% to 4% by weight, more preferably from about 1% to 3% by weight.

The anionic surfactant-containing powder produced by the drying operation constitutes two distinct phases, one of which is soluble in the inorganic liquid diluents used herein and one of which is insoluble in the diluents. The insoluble phase in the anionic surfactant-containing powder generally comprises from about 10% to 45% by weight of the powder, more preferably from about 15% to 35% by weight of a powder.

The anionic surfactant-containing powder that results after drying can comprise from about 45% to 94%, more preferably from about 60% to 94%, by weight of the powder of alkyl benzene sulfonic acid salts. Such concentrations are generally sufficient to provide from about 0.5% to 60%, more preferably from about 15% to 60%, by weight of the total detergent composition that is eventually prepared, of the alkyl benzene sulfonic acid salts. The anionic surfactant-containing powder itself can comprise from about 0.45% to 45% by weight of the total composition that is eventually prepared. After drying, the anionic surfactant-containing powder will also generally contain from about 2% to 50%, more preferably from about 2% to 25% by weight of the powder of the non-surfactant salts.

After it is dried to the requisite extent, the combined LAS/salt material can be converted to flakes or powder form by any known suitable milling or comminution process. Generally at the time such material is combined with the non-aqueous organic solvents to form the structured liquid phase of the compositions herein, the particle size of this powder will range from 0.1 to 2000 microns, more preferably from about 0.1 to 1000 microns.

A structured, surfactant-containing liquid phase of the preferred detergent compositions herein can be prepared by combining the non-aqueous organic diluents hereinbefore described with the anionic surfactant-containing powder as hereinbefore described. Such combination results in the formation of a structured surfactant-containing liquid phase. Conditions for making this combination of preferred structured liquid phase components are described more fully hereinafter in the “Composition Preparation and Use” section. As previously noted, the formation of a structured, surfactant-containing liquid phase permits the stable suspension of colored speckles and additional functional particulate solid materials within the preferred detergent compositions of this invention.

Additional suitable surfactants for use in the present invention included nonionic surfactants, specifically, polyhydroxy fatty acid amides of the formula:

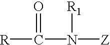

wherein R is a C₉₋₁₇ alkyl or alkenyl, R₁ is a methyl group and Z is glycityl derived from a reduced sugar or alkoxylated derivative thereof. Examples are N-methyl N-1-deoxyglucityl cocoamide and N-methyl N-1-deoxyglucityl oleamide. Processes for making polyhydroxy fatty acid amides are known and can be found in Wilson, U.S. Pat. No. 2,965,576 and Schwartz, U.S. Pat. No. 2,703,798, the disclosures of which are incorporated herein by reference.

Preferred surfactants for use in the detergent compositions described herein are amine based surfactants of the general formula:

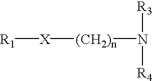

wherein R₁ is a C₆-C₁₂ alkyl group; n is from about 2 to about 4, X is a bridging group which is selected from NH, CONH, COO, or O or X can be absent; and R₃ and R₄ are individually selected from H, C₁-C₄ alkyl, or (CH₂—CH₂—O(R₅)) wherein R₅ is H or methyl. Especially preferred amines based surfactants include the following:

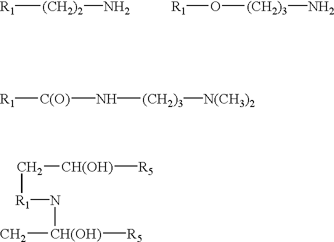

wherein R₁ is a C₆-C₁₂ alkyl group and R₅ is H or CH₃. Particularly preferred amines for use in the surfactants defined above include those selected from the group consisting of octyl amine, hexyl amine, decyl amine, dodecyl amine, C₈-C₁₂ bis(hydroxyethyl)amine, C₈-C₁₂ bis(hydroxyisopropyl)amine, C₈-C₁₂ amido-propyl dimethyl amine, or mixtures thereof.

In a highly preferred embodiment, the amine based surfactant is described by the formula:

R₁—C(O)—NH—(CH₂)₃—N(CH₃)₂

wherein R₁ is C₈-C₁₂ alkyl.

### Solid Particulate Materials

The non-aqueous detergent compositions herein preferably comprise from about 0.01% to 50% by weight, more preferably from about 0.2% to 30% by weight, of solid phase particulate material which is dispersed and suspended within the liquid phase. Generally such particulate material will range in size from about 0.1 to 1500 microns, more preferably from about 0.1 to 900 microns. Most preferably, such material will range in size from about 5 to 200 microns.

The particulate material utilized herein can comprise one or more types of detergent composition components which in particulate form are substantially insoluble in the non-aqueous liquid phase of the composition. The types of particulate materials which can be utilized are described in detail as follows:

Peroxygen Bleaching Acent With Optional Bleach Activators

The most preferred type of particulate material useful in the detergent compositions herein comprises particles of a peroxygen bleaching agent. Such peroxygen bleaching agents may be organic or inorganic in nature. Inorganic peroxygen bleaching agents are frequently utilized in combination with a bleach activator.

Useful organic peroxygen bleaching agents include percarboxylic acid bleaching agents and salts thereof. Suitable examples of this class of agents include magnesium monoperoxyphthalate hexahydrate, the magnesium salt of metachloro perbenzoic acid, 4-nonylamino-4-oxoperoxybutyric acid and diperoxydodecanedioic acid. Such bleaching agents are disclosed in U.S. Pat. No. 4,483,781, Hartman, Issued Nov. 20, 1984; European Patent Application EP-A-133,354, Banks et al., Published Feb. 20, 1985; and U.S. Pat. No. 4,412,934, Chung et al., Issued Nov. 1, 1983. Highly preferred bleaching agents also include 6-nonylamino-6-oxoperoxycaproic acid (NAPM) as described in U.S. Pat. No. 4,634,551, Issued Jan. 6, 1987 to Burns et al.

Inorganic peroxygen bleaching agents may also be used in particulate form in the detergent compositions herein. Inorganic bleaching agents are in fact preferred. Such inorganic peroxygen compounds include alkali metal perborate and percarbonate materials, most preferably the percarbonates. For example, sodium perborate (e.g. mono- or tetra-hydrate) can be used. Suitable inorganic bleaching agents can also include sodium or potassium carbonate peroxyhydrate and equivalent “percarbonate” bleaches, sodium pyrophosphate peroxyhydrate, urea peroxyhydrate, and sodium peroxide. Persulfate bleach (e.g., OXONE, manufactured commercially by DuPont) can also be used. Frequently inorganic peroxygen bleaches will be coated with silicate, borate, sulfate or water-soluble surfactants. For example, coated percarbonate particles are available from various commercial sources such as FMC, Solvay Interox, Tokai Denka and Degussa.

Inorganic peroxygen bleaching agents, e.g., the perborates, the percarbonates, etc., are preferably combined with bleach activators, which lead to the in situ production in aqueous solution (i.e., during use of the compositions herein for fabric laundering/bleaching) of the peroxy acid corresponding to the bleach activator. Various non-limiting examples of activators are disclosed in U.S. Pat. No. 4,915,854, Issued Apr. 10, 1990 to Mao et al.; and U.S. Pat. No. 4,412,934 Issued Nov. 1, 1983 to Chung et al. The nonanoyloxybenzene sulfonate (NOBS) and tetraacetyl ethylene diamine (TAED) activators are typical.

Mixtures thereof can also be used. See also the hereinbefore referenced U.S. Pat. No. 4,634,551 for other typical bleaches and activators useful herein.

Other useful amido-derived bleach activators are those of the formulae:

R¹N(R⁵)C(O)R²C(O)L or R¹C(O)N(R⁵)R²C(O)L

wherein R¹ is an alkyl group containing from about 6 to about 12 carbon atoms, R² is an alkylene containing from 1 to about 6 carbon atoms, R⁵ is H or alkyl, aryl, or alkaryl containing from about 1 to about 10 carbon atoms, and L is any suitable leaving group, for example, oxybenzene sulfonate, —OOH, —OOM. A leaving group is any group that is displaced from the bleach activator as a consequence of the nucleophilic attack on the bleach activator by the perhydrolysis anion. A preferred leaving group is phenol sulfonate.

Preferred examples of bleach activators of the above formulae include (6-octanamido-caproyl)oxybenzenesulfonate, (6-nonanamidocaproyl) oxybenzenesulfonate, (6-decanamido-caproyl)oxybenzenesulfonate and mixtures thereof as described in the hereinbefore referenced U.S. Pat. No. 4,634,551. Such mixtures are characterized herein as (6-C₈-C₁₀ alkamido-caproyl)oxybenzenesulfonate.

Another class of useful bleach activators comprises the benzoxazin-type activators disclosed by Hodge et al. in U.S. Pat. No. 4,966, 723, Issued Oct. 30, 1990, incorporated herein by reference. A highly preferred activator of the benzoxazin-type is:

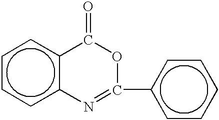

Still another class of useful bleach activators includes the acyl lactam activators, especially acyl caprolactams and acyl valerolactams of the formulae:

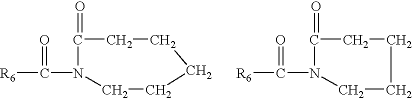

wherein R⁶ is H or an alkyl, aryl, alkoxyaryl, or alkaryl group containing from 1 to about 12 carbon atoms. Highly preferred lactam activators include benzoyl caprolactam, octanoyl caprolactam, 3,5,5-trimethylhexanoyl-caprolactam, nonanoyl caprolactam, decanoyl caprolactam, undecenoyl caprolactam, benzoyl valerolactam, octanoyl valerolactam, decanoyl valerolactam, undecenoyl valerolactam, 3,5,5-trimethylhexanoyl valerolactam and mixtures thereof. See also U.S. Pat. No. 4,545,784, Issued to Sanderson, Oct. 8, 1985, incorporated herein by reference, which discloses acyl caprolactams, including benzoyl caprolactam, adsorbed into sodium perborate.

If peroxygen bleaching agents are used as all or part of the particulate material, they will generally comprise from about 0.1% to 30% by weight of the composition. More. preferably, peroxygen bleaching agent will comprise from about 1% to 20% by weight of the composition. Most preferably, peroxygen bleaching agent will be present to the extent of from about 5% to 20% by weight of the composition. If utilized, bleach activators can comprise from about 0.5% to 20%, more preferably from about 3% to 10%, by weight of the composition. Frequently, activators are employed such that the molar ratio of bleaching agent to activator ranges from about 1:1 to 10:1, more preferably from about 1.5:1 to 5:1.

In addition, it has been found that bleach activators, when agglomerated with certain acids such as citric acid, are more chemically stable.

Organic Builder Material

Another possible type of particulate material which can be suspended in the non-aqueous liquid detergent compositions herein comprises an organic detergent builder material which serves to counteract the effects of calcium, or other ion, water hardness encountered during laundering/bleaching use of the compositions herein. Examples of such materials include the alkali metal, citrates, succinates, malonates, fatty acids, carboxymethyl succinates, carboxylates, polycarboxylates and polyacetyl carboxylates. Specific examples include sodium, potassium and lithium salts of oxydisuccinic acid, mellitic acid, benzene polycarboxylic acids and citric acid. Other examples of organic phosphonate type sequestering agents such as those which have been sold by Monsanto under the Dequest tradename and alkanehydroxy phosphonates. Citrate salts are highly preferred.

Other suitable organic builders include the higher molecular weight polymers and copolymers known to have builder properties. For example, such materials include appropriate polyacrylic acid, polymaleic acid, and polyacrylic/polymaleic acid copolymers and their salts, such as those sold by BASF under the Sokalan trademark which have molecular weight ranging from about 5,000 to 100,000.

Another suitable type of organic builder comprises the water-soluble salts of higher fatty acids, i.e., “soaps”. These include alkali metal soaps such as the sodium, potassium, ammonium, and alkylolammonium salts of higher fatty acids containing from about 8 to about 24 carbon atoms, and preferably from about 12 to about 18 carbon atoms. Soaps can be made by direct saponification of fats and oils or by the neutralization of free fatty acids. Particularly useful are the sodium and potassium salts of the mixtures of fatty acids derived from coconut oil and tallow, i.e., sodium or potassium tallow and coconut soap.

If utilized as all or part of the particulate material, insoluble organic detergent builders can generally comprise from about 2% to 20% by weight of the compositions herein. More preferably, such builder material can comprise from about 4% to 10% by weight of the composition.

Inorganic Alkalinity Sources

Another possible type of particulate material which can be suspended in the non-aqueous liquid detergent compositions herein can comprise a material which serves to render aqueous washing solutions formed from such compositions generally alkaline in nature. Such materials may or may not also act as detergent builders, i.e., as materials which counteract the adverse effect of water hardness on detergency performance.

Examples of suitable alkalinity sources include water-soluble alkali metal carbonates, bicarbonates, borates, silicates and metasilicates. Although not preferred for ecological reasons, water-soluble phosphate salts may also be utilized as alkalinity sources. These include alkali metal pyrophosphates, orthophosphates, polyphosphates and phosphonates. Of all of these alkalinity sources, alkali metal carbonates such as sodium carbonate are the most preferred.

The alkalinity source, if in the form of a hydratable salt, may also serve as a desiccant in the non-aqueous liquid detergent compositions herein. The presence of an alkalinity source which is also a desiccant may provide benefits in terms of chemically stabilizing those composition components such as the peroxygen bleaching agent which may be susceptible to deactivation by water.

If utilized as all or part of the particulate material component, the alkalinity source will generally comprise from about 1% to 25% by weight of the compositions herein. More preferably, the alkalinity source can comprise from about 2% to 15% by weight of the composition. Such materials, while water-soluble, will generally be insoluble in the non-aqueous detergent compositions herein. Thus such materials will generally be dispersed in the non-aqueous liquid phase in the form of discrete particles.

(D) Colored Speckles

The non-aqueous liquid detergent compositions herein also essentially contain from about 0.05% to 2%, more preferably 0.1% to 1%, of the composition of colored speckles. Such colored speckles themselves are combinations of a conventional dye or pigment material with a certain kind of carrier material that imparts specific characteristics to the speckles. For purposes of this invention, “colored” speckles are those which have a color that is visibly distinct from the color of the liquid detergent composition in which they are dispersed.

The colorant materials which can be used to form the colored speckles can comprise any of the conventional dyes and pigments known and approved for use in detergent products for use in the home. Such materials can include, for example, Ultramarine Blue dye, Acid 80 Blue dye, Red HP Liquitint, Blue Liquitint and the like.

Dye or pigment material can be combined with a specific type of carrier material to form the colored speckles for use in the detergent compositions herein. The carrier material is selected to impart to the speckles certain specific density and solubility characteristics. Materials which have been found to be suitable as carriers for the colored speckles include polyacrylates; polysaccharides such as starches, celluloses, gums and derivatives thereof; and polyethylene glycols. Especially preferred carrier material comprises polyethylene glycol having a molecular weight from about 4,000 to 20,000, more preferably from about 4,000 to 10,000.

The colored speckles can be produced by dispersing the dye or pigment material within the carrier material. This can be done, for example, by a) melting the carrier and dispersing the dye or pigment therein under mixing, b) mixing the dye/pigment powder and carrier powder together, or c) by dissolving the dye/pigment and the carrier in aqueous solution. The colorant/carrier mixture can then be formed into particles by flaking, spray drying, prilling, extruding or other conventional techniques. Generally the colored speckles will contain from about 0.1% to 5% by weight of the speckles of the colorant (dye or pigment) material.

The colored speckles produced in this manner will generally range in size from about 400 to 1,500 microns, more preferably from about 400 to 1,200 microns. Speckles made from the carrier materials specified will have a density less than about 1.4 g/cc, preferably from about 1.0 to 1.4 g/cc. Such speckles will also be substantially insoluble in the non-aqueous liquid phase of the liquid detergent compositions herein. Thus, the colored speckles can be stably suspended in the non-aqueous matrix of the liquid detergent compositions of this invention without dissolving therein. Such speckles, however, rapidly dissolve in the aqueous wash liquors prepared from the liquid detergent compositions herein.

### Aqueous Based Heavy Duty Liquid Detergents Surfactants

The present invention also comprises aqueous based liquid detergent compositions. The aqueous liquid detergent compositions preferably comprise from about 10% to about 98%, preferably from about 30% to about 95%, by weight of an aqueous liquid carrier which is preferably water. Additionally, the aqueous liquid detergent compositions of the present invention comprise a surfactant system which preferably contains one or more detersive surfactants. The surfactants can be selected from nonionic detersive surfactant, anionic detersive surfactant, zwitterionic detersive surfactant, amine oxide detersive surfactant, and mixtures thereof. The surfactant system typically comprises from about 5% to about 70%, preferably from about 15% to about 30%, by weight of the detergent composition.

Anionic Surfactant

Anionic surfactants include C₁₁-C₁₈ alkyl benzene sulfonates (LAS) and primary, branched-chain and random C₁₀-C₂₀ alkyl sulfates (AS), the C₁₀-C₁₈ secondary (2,3) alkyl sulfates of the formula CH₃(CH₂)ₓ((CHOSO₃—M⁺) CH₃ and CH₃ (CH₂)_{y}(CHOSO₃—M⁺) CH₂CH₃ where x and (y+1) are integers of at least about 7, preferably at least about 9, and M is a water-solubilizing cation, especially sodium, unsaturated sulfates such as oleyl sulfate, the C₁₀-C₁₈ alkyl alkoxy sulfates (“AEₓS”; especially EO 1-7 ethoxy sulfates), C₁₀-C₁₈ alkyl alkoxy carboxylates (especially the EO 1-5 ethoxycarboxylates), the C₁₀₋₁₈ glycerol ethers, the C₁₀-C₁₈ alkyl polyglycosides and their corresponding sulfated polyglycosides, and C₁₂-C₁₈ alpha-sulfonated fatty acid esters.

Generally speaking, anionic surfactants useful herein are disclosed in U.S. Pat. No. 4,285,841, Barrat et al, issued Aug. 25, 1981, and in U.S. Pat. No. 3,919,678, Laughlin et al, issued Dec. 30, 1975.

Useful anionic surfactants include the water-soluble salts, particularly the alkali metal, ammonium and alkylolammonium (e.g., monoethanolammonium or triethanolammonium) salts, of organic sulfuric reaction products having in their molecular structure an alkyl group containing from about 10 to about 20 carbon atoms and a sulfonic acid or sulfuric acid ester group. (Included in the term “alkyl” is the alkyl portion of aryl groups.) Examples of this group of synthetic surfactants are the alkyl sulfates, especially those obtained by sulfating the higher alcohols (C₈-C₁₈ carbon atoms) such as those produced by reducing the glycerides of tallow or coconut oil.

Other anionic surfactants herein are the water-soluble salts of alkyl phenol ethylene oxide ether sulfates containing from about 1 to about 4 units of ethylene oxide per molecule and from about 8 to about 12 carbon atoms in the alkyl group.

Other useful anionic surfactants herein include the water-soluble salts of esters of a-sulfonated fatty acids containing from about 6 to 20 carbon atoms in the fatty acid group and from about 1 to 10 carbon atoms in the ester group; water-soluble salts of 2-acyloxy-alkan-1-sulfonic acids containing from about 2 to 9 carbon atoms in the acyl group and from about 9 to about 23 carbon atoms in the alkane moiety; water-soluble salts of olefin sulfonates containing from about 12 to 24 carbon atoms; and b-alkyloxy alkane sulfonates containing from about 1 to 3 carbon atoms in the alkyl group and from about 8 to 20 carbon atoms in the alkane moiety.

Particularly preferred anionic surfactants herein are the alkyl polyethoxylate sulfates of the formula

RO(C₂H₄O)ₓSO₃—M⁺

wherein R is an alkyl chain having from about 10 to about 22 carbon atoms, saturated or unsaturated, M is a cation which makes the compound water-soluble, especially an alkali metal, ammonium or substituted ammonium cation, and x averages from about 1 to about 15.

Preferred alkyl sulfate surfactants are the non-ethoxylated C₁₂₋₁₅ primary and secondary alkyl sulfates. Under cold water washing conditions, i.e., less than abut 65° F. (18.3° C.), it is preferred that there be a mixture of such ethoxylated and non-ethoxylated alkyl sulfates. Examples of fatty acids include capric, lauric, myristic, palmitic, stearic, arachidic, and behenic acid. Other fatty acids include palmitoleic, oleic, linoleic, linolenic, and ricinoleic acid.

Nonionic Surfactant

Conventional nonionic and amphoteric surfactants include C₁₂-C₁₈ alkyl ethoxylates (AE) including the so-called narrow peaked alkyl ethoxylates and C₆-C₁₂ alkyl phenol alkoxylates (especially ethoxylates and mixed ethoxy/propoxy). The C₁₀-C₁₈ N-alkyl polyhydroxy fatty acid amides can also be used. Typical examples include the C₁₂-C₁₈ N-methylglucamides. See WO 9,206,154. Other sugar-derived surfactants include the N-alkoxy polyhydroxy fatty acid amides, such as C₁₀-C₁₈ N-(3-methoxypropyl) glucamide. Tbe N-propyl through N-hexyl C₁₂-C₁₈ glucamides can be used for low sudsing. C₁₀-C₂₀ conventional soaps may also be used. If high sudsing is desired, the branched-chain C₁₀-C₁₆ soaps may be used. Examples of nonionic surfactants are described in U.S. Pat. No. 4,285,841, Barrat et al, issued Aug. 25, 1981.

Preferred examples of these surfactants include ethoxylated alcohols and ethoxylated alkyl phenols of the formula R(OC₂H₄)ₙOH, wherein R is selected from the group consisting of aliphatic hydrocarbon radicals containing from about 8 to about 15 carbon atoms and alkyl phenyl radicals in which the alkyl groups contain from about 8 to about 12 carbon atoms, and the average value of n is from about 5 to about 15. These surfactants are more fully described in U.S. Pat. No. 4,284,532, Leikhim et al, issued Aug. 18, 1981. Particularly preferred are ethoxylated alcohols having an average of from about 10 to about 15 carbon atoms in the alcohol and an average degree of ethoxylation of from about 6 to about 12 moles of ethylene oxide per mole of alcohol. Mixtures of anionic and nonionic surfactants are especially useful.

Other conventional useful surfactants are listed in standard texts, including C₁₂-C₁₈ betaines and sulfobetaines (sultaines).

Amine Oxide Surfactants

The compositions herein also contain amine oxide surfactants of the formula:

R¹(EO)ₓ(PO)_{y}(BO)_{z}N(O)(CH₂R′)₂.qH₂O (I)

In general, it can be seen that the structure (I) provides one long-chain moiety R¹ (EO)ₓ(PO)_{y}(BO)_{z} and two short chain moieties, CH₂R′. R′ is preferably selected from hydrogen, methyl and —CH₂OH. In general R¹ is a primary or branched hydrocarbyl moiety which can be saturated or unsaturated, preferably, R¹ is a primary alkyl moiety. When x+y+z=0, R¹ is a hydrocarbyl moiety having chainlength of from about 8 to about 18. When x+y+z is different from 0, R¹ may be somewhat longer, having a chainlength in the range C₁₂-C₂₄. The general formula also encompasses amine oxides wherein x+y+z=0, R¹=C₈-C₁₈, R′ is H and q is 0-2, preferably 2. These amine oxides are illustrated by C₁₂₋₁₄ alkyldimethyl amine oxide, hexadecyl dimethylamine oxide, octadecylamine oxide and their hydrates, especially the dihydrates as disclosed in U.S. Pat. Nos. 5,075,501 and 5,071,594, incorporated herein by reference.

The invention also encompasses amine oxides wherein x+y+z is different from zero, specifically x+y+z is from about 1 to about 10, R¹ is a primary alkyl group containing 8 to about 24 carbons, preferably from about 12 to about 16 carbon atoms; in these embodiments y+z is preferably 0 and x is preferably from about 1 to about 6, more preferably from about 2 to about 4; EO represents ethyleneoxy; PO represents propyleneoxy; and BO represents butyleneoxy. Such amine oxides can be prepared by conventional synthetic methods, e.g., by the reaction of alkylethoxysulfates with dimethylamine followed by oxidation of the ethoxylated amine with hydrogen peroxide.

Highly preferred amine oxides herein are solids at ambient temperature, more preferably they have melting-points in the range 30° C. to 90° C. Amine oxides suitable for use herein are made commercially by a number of suppliers, including Akzo Chemie, Ethyl Corp., and Procter & Gamble. See McCutcheon's compilation and Kirk-Othmer review article for alternate amine oxide manufacturers. Preferred commercially available amine oxides are the solid, dihydrate ADMOX 16 and ADMOX 18, ADMOX 12 and especially ADMOX 14 from Ethyl Corp.

Preferred embodiments include dodecyldimethylamine oxide dihydrate, hexadecyldimethylamine oxide dihydrate, octadecyldimethylamine oxide dihydrate, hexadecyltris(ethyleneoxy)dimethyl-amine oxide, tetradecyldimethylamine oxide dihydrate, and mixtures thereof.

Whereas in certain of the preferred embodiments R′ is H, there is some latitude with respect to having R′ slightly larger than H. Specifically, the invention further encompasses embodiments wherein R′ is CH₂OH, such as hexadecylbis(2-hydroxyethyl)amine oxide, tallowbis(2-hydroxyethyl)amine oxide, stearylbis(2-hydroxyethyl)amine oxide and oleylbis(2-hydroxyethyl)amine oxide.

Builders

The compositions herein also optionally, but preferably, contain up to about 50%, more preferably from about 1% to about 40%, even more preferably from about 5% to about 30%, by weight of a detergent builder material. Lower or higher levels of builder, however, are not meant to be excluded. Detergent builders can optionally be included in the compositions herein to assist in controlling mineral hardness. Inorganic as well as organic builders can be used. Builders are typically used in fabric laundering compositions to assist in the removal of particulate soils. Detergent builders are described in U.S. Pat. No. 4,321,165, Smith et al, issued Mar. 23, 1982. Preferred builders for use in liquid detergents herein are described in U.S. Pat. No. 4,284,532, Leikhim et al, issued Aug. 18, 1981.

Examples of silicate builders are the alkali metal silicates, particularly those having a SiO₂:Na₂O ratio in the range 1.6:1 to 3.2:1 and layered silicates, such as the layered sodium silicates described in U.S. Pat. No. 4,664,839, issued May 12, 1987 to H. P. Rieck. NaSKS-6 is the trademark for a crystalline layered silicate marketed by Hoechst (commonly abbreviated herein as “SKS-6”). Unlike zeolite builders, the Na SKS-6 silicate builder does not contain aluminum. NaSKS-6 has the delta-Na₂SiO₅ morphology form of layered silicate. It can be prepared by methods such as those described in German DE-A-3,417,649 and DE-A-3,742,043. SKS-6 is a highly preferred layered silicate for use herein, but other such layered silicates, such as those having the general formula NaMSiₓO₂ₓ₊₁.yH₂O wherein M is sodium or hydrogen, x is a number from 1.9 to 4, preferably 2, and y is a number from 0 to 20, preferably 0 can be used herein. Various other layered silicates from Hoechst include NaSKS-5, NaSKS-7 and NaSKS-11, as the alpha, beta and gamma forms. As noted above, the delta-Na₂SiO₅ (NaSKS-6 form) is most preferred for use herein. Other silicates may also be useful such as for example magnesium silicate, which can serve as a stabilizing agent for oxygen bleaches and as a component of suds control systems.

Examples of carbonate builders are the alkaline earth and alkali metal carbonates as disclosed in German Patent Application No. 2,321,001 published on Nov. 15, 1973.

Aluminosilicate builders are useful in the present invention. Aluminosilicate builders can be a significant builder ingredient in liquid detergent formulations. Aluminosilicate builders include those having the empirical formula:

M_{z}(zAlO₂)_{y}].xH₂O

wherein z and y are integers of at least 6, the molar ratio of z to y is in the range from 1.0 to about 0.5, and x is an integer from about 15 to about 264.

Useful aluminosilicate ion exchange materials are commercially available. These aluminosilicates can be crystalline or amorphous in structure and can be naturally-occurring aluminosilicates or synthetically derived. A method for producing aluminosilicate ion exchange materials is disclosed in U.S. Pat. No. 3,985,669, Krummel, et al, issued Oct. 12, 1976. Preferred synthetic crystalline aluminosilicate ion exchange materials useful herein are available under the designations Zeolite A, Zeolite P (B), Zeolite MAP and Zeolite X. In an especially preferred embodiment, the crystalline aluminosilicate ion exchange material has the formula:

Na₁₂[(AlO₂)₁₂(SiO₂)₁₂].xH₂O

wherein x is from about 20 to about 30, especially about 27. This material is known as Zeolite A. Dehydrated zeolites (x=0-10) may also be used herein. Preferably, the aluminosilicate has a particle size of about 0.1-10 microns in diameter.

Organic detergent builders suitable for the purposes of the present invention include, but are not restricted to, a wide variety of polycarboxylate compounds. As used herein, “polycarboxylate” refers to compounds having a plurality of carboxylate groups, preferably at least 3 carboxylates. Polycarboxylate builder can generally be added to the composition in acid form, but can also be added in the form of a neutralized salt. When utilized in salt form, alkali metals, such as sodium, potassium, and lithium, or alkanolammonium salts are preferred.

Included among the polycarboxylate builders are a variety of categories of useful materials. One important category of polycarboxylate builders encompasses the ether polycarboxylates, including oxydisuccinate, as disclosed in Berg, U.S. Pat. No. 3,128,287, issued Apr. 7, 1964, and Lamberti et al, U.S. Pat. No. 3,635,830, issued Jan. 18, 1972. See also “TMS/TDS” builders of U.S. Pat. No. 4,663,071, issued to Bush et al, on May 5, 1987. Suitable ether polycarboxylates also include cyclic compounds, particularly alicyclic compounds, such as those described in U.S. Pat. No. 3,923,679; 3,835,163; 4,158,635; 4,120,874 and 4,102,903.

Other useful detergency builders include the ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, oxydisuccinic acid, polymaleic acid, benzene 1,3,5-tricarboxylic acid, carboxymethyloxysuccinic acid, and soluble salts thereof.

Citrate builders, e.g., citric acid and soluble salts thereof (particularly sodium salt), are polycarboxylate builders of particular importance-for heavy duty liquid detergent formulations due to their availability from renewable resources and their biodegradability. Oxydisuccinates are also especially useful in such compositions and combinations.

Also suitable in the detergent compositions of the present invention are the 3,3-dicarboxy-4-oxa-1,6-hexanedioates and the related compounds disclosed in U.S. Pat. No. 4,566,984, Bush, issued Jan. 28, 1986. Useful succinic acid builders include the C₅-C₂₀ alkyl and alkenyl succinic acids and salts thereof. A particularly preferred compound of this type is dodecenylsuccinic acid. Specific examples of succinate builders include: laurylsuccinate, myristylsuccinate, palmitylsuccinate, 2-dodecenylsuccinate (preferred), 2-pentadecenylsuccinate, and the like. Laurylsuccinates are the preferred builders of this group, and are described in European Patent Application 86200690.5/0,200,263, published Nov. 5, 1986.

Other suitable polycarboxylates are disclosed in U.S. Pat. No. 4,144,226, Crutchfield et al, issued Mar. 13, 1979 and in U.S. Pat. No. 3,308,067, Diehl, issued Mar. 7, 1967. See also Diehl U.S. Pat. No. 3,723,322.

Fatty acids, e.g., C₁₂-C₁₈ monocarboxylic acids, can also be incorporated into the compositions alone, or in combination with the aforesaid builders, especially citrate and/or the succinate builders, to provide additional builder activity. Such use of fatty acids will generally result in a diminution of sudsing, which should be taken into account by the formulator.

In situations where phosphorus-based builders can be used the various alkali metal phosphates such as the well-known sodium tripolyphosphates, sodium pyrophosphate and sodium orthophosphate can be used. Phosphonate builders such as ethane-1-hydroxy-1,1-diphosphonate and other known phosphonates (see, for example, U.S. Pat. NOs. 3,159,581; 3,213,030; 3,422,021; 3,400,148 and 3,422,137) can also be used.

### OTHER OPTIONAL COMPOSITION COMPONENTS

In addition to the liquid and solid phase components as hereinbefore described, the aqueous and non-aqueous based detergent compositions can, and preferably will, contain various other optional components. Such optional components may be in either liquid or solid form. The optional components may either dissolve in the liquid phase or may be dispersed within the liquid phase in the form of fine particles or droplets. Some of the other materials which may optionally be utilized in the compositions herein are described in greater detail as follows:

Optional Inorganic Detergent Builders

The detergent compositions herein may also optionally contain one or more types of inorganic detergent builders beyond those listed hereinbefore that also function as alkalinity sources. Such optional inorganic builders can include, for example, aluminosilicates such as zeolites. Aluminosilicate zeolites, and their use as detergent builders are more fully discussed in Corkill et al., U.S. Pat. No. 4,605,509; Issued Aug. 12, 1986, the disclosure of which is incorporated herein by reference. Also crystalline layered silicates, such as those discussed in this '509 U.S. patent, are also suitable for use in the detergent compositions herein. If utilized, optional inorganic detergent builders can comprise from about 2% to 15% by weight of the compositions herein.

Optional Enzymes

The detergent compositions herein may also optionally contain one or more types of detergent enzymes. Such enzymes can include proteases, amylases, cellulases and lipases. Such materials are known in the art and are commercially available. They may be incorporated into the non-aqueous liquid detergent compositions herein in the form of suspensions, “marumes” or “prills”. Another suitable type of enzyme comprises those in the form of slurries of enzymes in nonionic surfactants, e.g., the enzymes marketed by Novo Nordisk under the tradename “SL” or the microencapsulated enzymes marketed by Novo Nordisk under the tradename “LDP.”

Enzymes added to the compositions herein in the form of conventional enzyme prills are especially preferred for use herein. Such prills will generally range in size from about 100 to 1,000 microns, more preferably from about 200 to 800 microns and will be suspended throughout the non-aqueous liquid phase of the composition. Prills in the compositions of the present invention have been found, in comparison with other enzyme forms, to exhibit especially desirable enzyme stability in terms of retention of enzymatic activity over time. Thus, compositions which utilize enzyme prills need not contain conventional enzyme stabilizing such as must frequently be used when enzymes are incorporated into aqueous liquid detergents.

If employed, enzymes will normally be incorporated into the non-aqueous liquid compositions herein at levels sufficient to provide up to about 10 mg by weight, more typically from about 0.01 mg to about 5 mg, of active enzyme per gram of the composition. Stated otherwise, the non-aqueous liquid detergent compositions herein will typically comprise from about 0.001% to 5%, preferably from about 0.01% to 1% by weight, of a commercial enzyme preparation. Protease enzymes, for example, are usually present in such commercial preparations at levels sufficient to provide from 0.005 to 0.1 Anson units (AU) of activity per gram of composition.

Optional Chelating Agents

The detergent compositions herein may also optionally contain a chelating agent which serves to chelate metal ions, e.g., iron and/or manganese, within the non-aqueous detergent compositions herein. Such chelating agents thus serve to form complexes with metal impurities in the composition which would otherwise tend to deactivate composition components such as the peroxygen bleaching agent. Useful chelating agents can include amino carboxylates, phosphonates, amino phosphonates, polyfunctionally-substituted aromatic chelating agents and mixtures thereof.

Amino carboxylates useful as optional chelating agents include ethylenediaminetetraacetates, N-hydroxyethyl-ethylenediaminetriacetates, nitrilotriacetates, ethylene-diamine tetrapropionates, triethylenetetraaminehexacetates, diethylenetriaminepentaacetates, ethylenediaminedisuccinates and ethanol diglycines. The alkali metal salts of these materials are preferred.

Amino phosphonates are also suitable for use as chelating agents in the compositions of this invention when at least low levels of total phosphorus are permitted in detergent compositions, and include ethylenediaminetetrakis (methylene-phosphonates) as DEQUEST. Preferably, these amino phosphonates do not contain alkyl or alkenyl groups with more than about 6 carbon atoms.

Polyfunctionally-substituted aromatic chelating agents are also useful in the compositions herein. See U.S. Pat. No. 3,812,044, issued May 21, 1974, to Connor et al. Preferred compounds of this type in acid form are dihydroxydisulfobenzenes such as 1,2-dihydroxy-3,5-disulfobenzene.

Preferred chelating agents include hydroxy-ethyldiphosphonic acid (HEDP), diethylene triamine penta acetic acid (DTPA), ethylenediamine disuccinic acid (EDDS) and dipicolinic acid (DPA) and salts thereof. The chelating agent may, of course, also act as a detergent builder during use of the compositions herein for fabric laundering/bleaching. The chelating agent, if employed, can comprise from about 0.1% to 10% by weight of the compositions herein. More preferably, the chelating agent will comprise from about 0.2% to 3% by weight of the detergent compositions herein.

Optional Clay Soil Removal/Anti-Redeposition Agents

The compositions of the present invention can also optionally contain water-soluble ethoxylated amines having clay soil removal and anti-redeposition properties. If used, soil materials can contain from about 0.01% to about 5% by weight of the compositions herein.

The most preferred soil release and anti-redeposition agent is ethoxylated tetraethylenepentamine. Exemplary ethoxylated amines are further described in U.S. Pat. No. 4,597,898, VanderMeer, issued Jul. 1, 1986. Another group of preferred clay soil removal-anti-redeposition agents are the cationic compounds disclosed in European Patent Application 111,965, Oh and Gosselink, published Jun. 27, 1984. Other clay soil removal/anti-redeposition agents which can be used include the ethoxylated amine polymers disclosed in European Patent Application 111,984, Gosselink, published Jun. 27, 1984; the zwitterionic polymers disclosed in European Patent Application 112,592, Gosselink, published Jul. 4, 1984; and the amine oxides disclosed in U.S. Pat. No. 4,548,744, Connor, issued Oct. 22, 1985. Other clay soil removal and/or anti-redeposition agents known in the art can also be utilized in the compositions herein. Another type of preferred anti-redeposition agent includes the carboxy methyl cellulose (CMC) materials. These materials are well known in the art.

Optional Liquid Bleach Activators

The detergent compositions herein may also optionally contain bleach activators which are liquid in form at room temperature and which can be added as liquids to the non-aqueous liquid phase of the detergent compositions herein. One such liquid bleach activator is acetyl triethyl citrate (ATC). Other examples include glycerol triacetate and nonanoyl valerolactam. Liquid bleach activators can be dissolved in the non-aqueous liquid phase of the compositions herein.

Optional Bleach Catalysts

If desired, the bleaching compounds can be catalyzed by means of a manganese compound. Such compounds are well known in the art and include, for example, the manganese-based catalysts disclosed in U.S. Pat. Nos. 5,246,621, 5,244,594; 5,194,416; 5,114,606; and European Pat. App. Pub. Nos. 549,271A1, 549,272A1, 544,440A2, and 544,490A1; Preferred examples of these catalysts include Mn^{IV}₂(u-O)₃(1,4,7-trimethyl-1,4,7-triazacyclononane)₂(PF₆)₂, Mn^{III}₂(u-O)₁(u-OAc)₂(1,4,7-trimethyl-1,4,7-triazacyclononane)₂(ClO₄)₂, Mn^{IV}₄(u-O)₆(1,4,7-triazacyclononane)₄(ClO₄)₄, Mn^{III}Mn^{IV}₄(u-O)₁ (u-OAc)₂-(1,4,7-trimethyl-1,4,7-triazacyclononane)₂(ClO₄)₃, Mn^{IV}(1,4,7-trimethyl-1,4,7-triazacyclononane)-(OCH₃)₃(PF₆), and mixtures thereof. Other metal-based bleach catalysts include those disclosed in U.S. Pat. Nos. 4,430,243 and 5,114,611. The use of manganese with various complex ligands to enhance bleaching is also reported in the following U.S. pat. Nos. 4,728,455; 5,284,944; 5,246,612; 5,256,779; 5,280,117; 5,274,147; 5,153,161; and 5,227,084.

As a practical matter, and not by way of limitation, the compositions and processes herein can be adjusted to provide on the order of at least one part per ten million of the active bleach catalyst species in the aqueous washing liquor, and will preferably provide from about 0.1 ppm to about 700 ppm, more preferably from about 1 ppm to about 500 ppm, of the catalyst species in the laundry liquor.

Cobalt bleach catalysts useful herein are known, and are described, for example, in M. L. Tobe, “Base Hydrolysis of Transition-Metal Complexes”, Adv. Inorg. Bioinorg. Mech., (1983), 2, pages 1-94. The most preferred cobalt catalyst useful herein are cobalt pentaamine acetate salts having the formula [Co(NH₃)₅OAc] T_{y}, wherein “OAc” represents an acetate moiety and “T_{y}” is an anion, and especially cobalt pentaamine acetate chloride, [Co(NH₃)₅OAc]Cl₂; as well as [Co(NH₃)₅OAc](OAc)₂; [Co(NH₃)₅OAc](PF₆)₂; [Co(NH₃)₅OAc](SO₄); [Co(NH₃)₅OAc](BF₄)₂; and [Co(NH₃)₅OAc](NO₃)₂ (herein “PAC”).

These cobalt catalysts are readily prepared by known procedures, such as taught for example in the Tobe article and the references cited therein, in U.S. Pat. No. 4,810,410, to Diakun et al, issued Mar. 7,1989, J. Chem. Ed. (1989), 66 (12), 1043-45; The Synthesis and Characterization of Inorganic Compounds, W. L. Jolly (Prentice-Hall; 1970), pp. 461-3; Inorg. Chem., 18, 1497-1502 (1979); Inorg. Chem., 21, 2881-2885 (1982); Inorg. Chem., 18, 2023-2025 (1979); Inorg. Synthesis, 173-176 (1960); and Journal of Physical Chemistry, 56, 22-25 (1952).

As a practical matter, and not by way of limitation, the compositions and cleaning processes herein can be adjusted to provide on the order of at least one part per hundred million of the active bleach catalyst species in the aqueous washing medium, and will preferably provide from about 0.01 ppm to about 25 ppm, more preferably from about 0.05 ppm to about 10 ppm, and most preferably from about 0.1 ppm to about 5 ppm, of the bleach catalyst species in the wash liquor. In order to obtain such levels in the wash liquor of an automatic washing process, typical compositions herein will comprise from about 0.0005% to about 0.2%, more preferably from about 0.004% to about 0.08%, of bleach catalyst, especially manganese or cobalt catalysts, by weight of the cleaning compositions.

Optional Brighteners, Suds Suppressors, Dyes and/or Perfumes

The detergent compositions herein may also optionally contain conventional brighteners, suds suppressors, dyes and/or perfume materials. Such brighteners, suds suppressors, silicone oils, dyes and perfumes must, of course, be compatible and non-reactive with the other composition components in a non-aqueous environment. If present, brighteners suds suppressors, dyes and/or perfumes will typically comprise from about 0.0001% to 2% by weight of the compositions herein.

Structure Elasticizing Agents

The non-aqueous liquid detergent compositions herein can also contain from about 0.1% to 5%, preferably from about 0.1% to 2% by weight of a finely divided, solid particulate material which can include silica, e.g., fumed silica, titanium dioxide, insoluble carbonates, finely divided carbon or combinations of these materials. Fine particulate material of this type functions as a structure elasticizing agent in the products of this invention. Such material has an average particle size ranging from about 7 to 40 nanometers, more preferably from about 7 to 15 nanometers. Such material also has a specific surface area which ranges from about 40 to 400 m²/g.

The finely divided elasticizing agent material can improve the shipping stability of the non-aqueous liquid detergent products herein by increasing the elasticity of the surfactant-structured liquid phase without increasing product viscosity. This permits such products to withstand high frequency vibration which may be encountered during shipping without undergoing undesirable structure breakdown which could lead to sedimentation in the product.

In the case of titanium dioxide, the use of this material also imparts whiteness to the suspension of particulate material within the detergent compositions herein. This effect improves the overall appearance of the product.

Polymeric Soil Release Agent

Any polymeric soil release agent known to those skilled in the art can optionally be employed in the compositions and processes of this invention. Polymeric soil release agents are characterized by having both hydrophilic segments, to hydrophilize the surface of hydrophobic fibers, such as polyester and nylon, and hydrophobic segments, to deposit upon hydrophobic fibers and remain adhered thereto through completion of washing and rinsing cycles and, thus, serve as an anchor for the hydrophilic segments. This can enable stains occurring subsequent to treatment with the soil release agent to be more easily cleaned in later washing procedures.

Examples of polymeric soil release agents useful herein include U.S. Pat. No. 4,721,580, issued Jan. 26, 1988 to Gosselink; U.S. Pat. No. 4,000,093, issued Dec. 28, 1976 to Nicol, et al.; European Patent Application 0 219 048, published Apr. 22, 1987 by Kud, et al.; U.S. Pat. No. 4,702,857, issued Oct. 27, 1987 to Gosselink; U.S. Pat. No. 4,968,451, issued Nov. 6, 1990 to J. J. Scheibel. Commercially available soil release agents include the SOKALAN type of material, e.g., SOKALAN HP-22, available from BASF (West Germany). Also see U.S. Pat. No. 3,959,230 to Hays, issued May 25, 1976 and U.S. Pat. No. 3,893,929 to Basadur issued Jul. 8, 1975. Examples of this polymer include the commercially available material ZELCON 5126 (from Dupont) and MILEASE T (from ICI). Other suitable polymeric soil release agents include the terephthalate polyesters of U.S. Pat. No. 4,711,730, issued Dec. 8, 1987 to Gosselink et al, the anionic end-capped oligomeric esters of U.S. Pat. No. 4,721,580, issued Jan. 26, 1988 to Gosselink, and the block polyester oligomeric compounds of U.S. Pat. No. 4,702,857, issued Oct. 27, 1987 to Gosselink. Preferred polymeric soil release agents also include the soil release agents of U.S. Pat. No. 4,877,896, issued Oct. 31, 1989 to Maldonado et al.

If utilized, soil release agents will generally comprise from about 0.01% to about 10.0%, by weight, of the detergent compositions herein, typically from about 0.1% to about 5%, preferably from about 0.2% to about 3.0% by weight of the detergent compositions.

Polymeric Dispersing Agents

Polymeric dispersing agents can advantageously be utilized at levels from about 0.1% to about 7%, by weight, in the compositions herein, especially in the presence of zeolite and/or layered silicate builders. Suitable polymeric dispersing agents include polymeric polycarboxylates and polyethylene glycols, although others known in the art can also be used. It is believed, though it is not intended to be limited by theory, that polymeric dispersing agents enhance overall detergent builder performance, when used in combination with other builders (including lower molecular weight polycarboxylates) by crystal growth inhibition, particulate soil release peptization, and anti-redeposition.

Polymeric polycarboxylate materials can be prepared by polymerizing or copolymerizing suitable unsaturated monomers, preferably in their acid form. Unsaturated monomeric acids that can be polymerized to form suitable polymeric polycarboxylates include acrylic acid, maleic acid (or maleic anhydride), fumaric acid, itaconic acid, aconitic acid, mesaconic acid, citraconic acid and methylenemalonic acid. The presence in the polymeric polycarboxylates herein or monomeric segments, containing no carboxylate radicals such as vinylmethyl ether, styrene, ethylene, etc. is suitable provided that such segments do not constitute more than about 40% by weight.

Particularly suitable polymeric polycarboxylates can be derived from acrylic acid. Such acrylic acid-based polymers which are useful herein are the water-soluble salts of polymerized acrylic acid. The average molecular weight of such polymers in the acid form preferably ranges from about 2,000 to 10,000, more preferably from about 4,000 to 7,000 and most preferably from about 4,000 to 5,000. Water-soluble salts of such acrylic acid polymers can include, for example, the alkali metal, ammonium and substituted ammonium salts. Soluble polymers of this type are known materials. Use of polyacrylates of this type in detergent compositions has been disclosed, for example, in Diehl, U.S. Pat. No. 3,308,067, issued mar. 7, 1967.

Acrylic/maleic-based copolymers may also be used as a preferred component of the dispersing/anti-redeposition agent. Such materials include the water-soluble salts of copolymers of acrylic acid and maleic acid. The average molecular weight of such copolymers in the acid form preferably ranges from about 2,000 to 100,000, more preferably from about 5,000 to 75,000, most preferably from about 7,000 to 65,000. The ratio of acrylate to maleate segments in such copolymers will generally range from about 30:1 to about 1:1, more preferably from about 10:1 to 2:1. Water-soluble salts of such acrylic acid/maleic acid copolymers can include, for example, the alkali metal, ammonium and substituted ammonium salts. Soluble acrylate/maleate copolymers of this type are known materials which are described in European Patent Application No. 66915, published Dec. 15, 1982, as well as in EP 193,360, published Sep. 3, 1986, which also describes such polymers comprising hydroxypropylacrylate. Still other useful dispersing agents include the maleic/acrylic/vinyl alcohol terpolymers. Such materials are also disclosed in EP 193,360, including, for example, the 45/45/10 terpolymer of acrylic/maleic/vinyl alcohol.

Another polymeric material which can be included is polyethylene glycol (PEG). PEG can exhibit dispersing agent performance as well as act as a clay soil removal-antiredeposition agent. Typical molecular weight ranges for these purposes range from about 500 to about 100,000, preferably from about 1,000 to about 50,000, more preferably from about 1,500 to about 10,000.

Polyaspartate and polyglutamate dispersing agents may also be used, especially in conjunction with zeolite builders. Dispersing agents such as polyaspartate preferably have a molecular weight (avg.) of about 10,000.

Dye Transfer Inhibiting Agents

The compositions of the present invention may also include one or more materials effective for inhibiting the transfer of dyes from one fabric to another during the cleaning process. Generally, such dye transfer inhibiting agents include polyvinyl pyrrolidone polymers, polyamine N-oxide polymers, copolymers of N-vinylpyrrolidone and N-vinylimidazole, manganese phthalocyanine, peroxidases, and mixtures thereof. If used, these agents typically comprise from about 0.01% to about 10% by weight of the composition, preferably from about 0.01% to about 5%, and more preferably from about 0.05% to about 2%.

More specifically, the polyamine N-oxide polymers preferred for use herein contain units having the following structural formula: R—Aₓ—P; wherein P is a polymerizable unit to which an N—O group can be attached or the N—O group can form part of the polymerizable unit or the N—O group can be attached to both units; A is one of the following structures: —NC(O)—, —C(O)O—, —S—, —O—, —N=; x is 0 or 1; and R is aliphatic, ethoxylated aliphatics, aromatics, heterocyclic or alicyclic groups or any combination thereof to which the nitrogen of the N—O group can be attached or the N—O group is part of these groups. Preferred polyamine N-oxides are those wherein R is a heterocyclic group such as pyridine, pyrrole, imidazole, pyrrolidine, piperidine and derivatives thereof.

The N—O group can be represented by the following general structures:

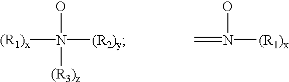

wherein R₁, R₂, R₃ are aliphatic, aromatic, heterocyclic or alicyclic groups or combinations thereof; x, y and z are 0 or 1; and the nitrogen of the N—O group can be attached or form part of any of the aforementioned groups. The amine oxide unit of the polyamine N-oxides has a pKa <10, preferably pKa <7, more preferred pKa <6.

Any polymer backbone can be used as long as the amine oxide polymer formed is water-soluble and has dye transfer inhibiting properties: Examples of suitable polymeric backbones are polyvinyls, polyalkylenes, polyesters, polyethers, polyamide, polyimides, polyacrylates and mixtures thereof. These polymers include random or block copolymers where one monomer type is an amine N-oxide and the other monomer type is an N-oxide. The amine N-oxide polymers typically have a ratio of amine to the amine N-oxide of 10:1 to 1:1,000,000. However, the number of amine oxide groups present in the polyamine oxide polymer can be varied by appropriate copolymerization or by an appropriate degree of N-oxidation. The polyamine oxides can be obtained in almost any degree of polymerization. Typically, the average molecular weight is within the range of 500 to 1,000,000; more preferred 1,000 to 500,000; most preferred 5,000 to 100,000. This preferred class of materials can be referred to as “PVNO”.

The most preferred polyamine N-oxide useful in the detergent compositions herein is poly(4-vinylpyridine-N-oxide) which as an average molecular weight of about 50,000 and an amine to amine N-oxide ratio of about 1:4.

Copolymers of N-vinylpyrrolidone and N-vinylimidazole polymers (referred to as a class as “PVPVI”) are also preferred for use herein. Preferably the PVPVI has an average molecular weight range from 5,000 to 1,000,000, more preferably from 5,000 to 200,000, and most preferably from 10,000 to 20,000. (The average molecular weight range is determined by light scattering as described in Barth, et al., Chemical Analysis, Vol 113. “Modem Methods of Polymer Characterization”, the disclosures of which are incorporated herein by reference.) The PVPVI copolymers typically have a molar ratio of N-vinylimidazole to N-vinylpyrrolidone from 1:1 to 0.2:1, more preferably from 0.8:1 to 0.3:1, most preferably from 0.6:1 to 0.4:1. These copolymers can be either linear or branched.

The present invention compositions also may employ a polyvinylpyrrolidone (“PVP”) having an average molecular weight of from about 5,000 to about 400,000, preferably from about 5,000 to about 200,000, and more preferably from about 5,000 to about 50,000. PVP's are known to persons skilled in the detergent field; see, for example, EP-A-262,897 and EP-A-256,696, incorporated herein by reference. Compositions containing PVP can also contain polyethylene glycol (“PEG”) having an average molecular weight from about 500 to about 100,000, preferably from about 1,000 to about 10,000. Preferably, the ratio of PEG to PVP on a ppm basis delivered in wash solutions is from about 2:1 to about 50:1, and more preferably from about 3:1 to about 10:1.

The detergent compositions herein may also optionally contain from about 0.005% to 5% by weight of certain types of hydrophilic optical brighteners which also provide a dye transfer inhibition action. If used, the compositions herein will preferably comprise from about 0.01% to 1% by weight of such optical brighteners.

The hydrophilic optical brighteners useful in the present invention are those having the structural formula:

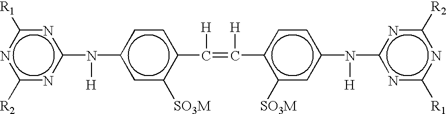

wherein R₁ is selected from anilino, N-2-bis-hydroxyethyl and NH-2-hydroxyethyl; R₂ is selected from N-2-bis-hydroxyethyl, N-2-hydroxyethyl-N-methylamino, morphilino, chloro and amino; and M is a salt-forming cation such as sodium or potassium.

When in the above formula, R₁ is anilino, R₂ is N-2-bis-hydroxyethyl and M is a cation such as sodium, the brightener is 4,4′,-bis[(4-anilino-6-(N-2-bis-hydroxyethyl)-s-triazine-2-yl)amino]-2,2′-stilbenedisulfonic acid and disodium salt. This particular brightener species is commercially marketed under the tradename Tinopal-UNPA-GX by Ciba-Geigy Corporation. Tinopal-UNPA-GX is the preferred hydrophilic optical brightener useful in the detergent compositions herein.

When in the above formula, R₁ is anilino, R₂ is N-2-hydroxyethyl-N-2-methylamino and M is a cation such as sodium, the brightener is 4,4′-bis[(4-anilino-6-(N-2-hydroxyethyl-N-methylamino)-s-triazine-2-yl)amino]2,2′-stilbenedisulfonic acid disodium salt. This particular brightener species is commercially marketed under the tradename Tinopal 5BM-GX by Ciba-Geigy Corporation.

When in the above formula, R₁ is anilino, R₂ is morphilino and M is a cation such as sodium, the brightener is 4,4′-bis[(4-anilino-6-morphilino-s-triazine-2-yl)amino]2,2′-stilbenedisulfonic acid, sodium salt. This particular brightener species is commercially marketed under the tradename Tinopal AMS-GX by Ciba Geigy Corporation.

The specific optical brightener species selected for use in the present invention provide especially effective dye transfer inhibition performance benefits when used in combination with the selected polymeric dye transfer inhibiting agents hereinbefore described. The combination of such selected polymeric materials (e.g., PVNO and/or PVPVI) with such selected optical brighteners (e.g., Tinopal UNPA-GX, Tinopal 5BM-GX and/or Tinopal AMS-GX) provides significantly better dye transfer inhibition in aqueous wash solutions than does either of these two detergent composition components when used alone. Without being bound by theory, it is believed that such brighteners work this way because they have high affinity for fabrics in the wash solution and therefore deposit relatively quick on these fabrics. The extent to which brighteners deposit on fabrics in the wash solution can be defined by a parameter called the “exhaustion coefficient”. The exhaustion coefficient is in general as the ratio of a) the brightener material deposited on fabric to b) the initial brightener concentration in the wash liquor. Brighteners with relatively high exhaustion coefficients are the most suitable for inhibiting dye transfer in the context of the present invention.

Of course, it will be appreciated that other, conventional optical brightener types of compounds can optionally be used in the present compositions to provide conventional fabric “brightness” benefits, rather than a true dye transfer inhibiting effect. Such usage is conventional and well-known to detergent formulations.

### Composition Form

As indicated, the non-aqueous liquid detergent compositions herein are in the form of bleaching agent and/or other materials in particulate form as a solid phase suspended in and dispersed throughout a surfactant-containing, preferably structured non-aqueous liquid phase. Generally, the structured non-aqueous liquid phase will comprise from about 45% to 95%, more preferably from about 50% to 90%, by weight of the composition with the dispersed additional solid materials comprising from about 5% to 55%, more preferably from about 10% to 50%, by weight of the composition.

The particulate-containing liquid detergent compositions of this invention are substantially non-aqueous (or anhydrous) in character. While very small amounts of water may be incorporated into such compositions as an impurity in the essential or optional components, the amount of water should in no event exceed about 5% by weight of the compositions herein. More preferably, water content of the non-aqueous detergent compositions herein will comprise less than about 1% by weight.

The particulate-containing non-aqueous liquid detergent compositions herein will be relatively viscous and phase stable under conditions of commercial marketing and use of such compositions. Frequently the viscosity of the compositions herein will range from about 300 to 5,000 cps, more preferably from about 500 to 3,000 cps. For purposes of this invention, viscosity is measured with a Carrimed CSL2 Rheometer at a shear rate of 20 s⁻¹.

The aqueous based heavy-duty liquid detergent compositions disclosed herein can contain water and other solvents as carriers. Low molecular weight primary or secondary alcohols exemplified by methanol, ethanol, propanol, and isopropanol are suitable. Monohydric alcohols are preferred for solubilizing surfactant, but polyols such as those containing from 2 to about 6 carbon atoms and from 2 to about 6 hydroxy groups (e.g., 1,3-propanediol, ethylene glycol, glycerine, and 1,2-propanediol) can also be used. The compositions may contain from 5% to 90%, typically 10% to 50% of such carriers.

The detergent compositions herein will preferably be formulated such that, during use in aqueous cleaning operations, the wash water will have a pH of between about 6.5 and about 11, preferably between about 7.5 and 11. Techniques for controlling pH at recommended usage levels include the use of buffers, alkalis, acids, etc., and are well known to those skilled in the art.

### Composition Preparation and Use

The aqueous based heavy-duty liquid detergent compositions of the present invention can be made by mixing and blending the desired ingredients with the desired sovent. The non-aqueous liquid detergent compositions herein can be prepared by first forming the surfactant-containing, preferably structured non-aqueous liquid phase and by thereafter adding to this structured phase the particulate components in any convenient order and by mixing, e.g., agitating, the resulting component combination to form the phase stable compositions herein. In a typical process for preparing such compositions, essential and certain preferred optional components will be combined in a particular order and under certain conditions.

In a first step of a preferred preparation process, the anionic surfactant-containing powder used to form the structured, surfactant-containing liquid phase is prepared. This pre-preparation step involves the formation of an aqueous slurry containing from about 30% to 60% of one or more alkali metal salts of linear C₁₀₋₁₆ alkyl benzene sulfonic acid and from about 2% to 30% of one or more diluent non-surfactant salts. In a subsequent step, this slurry is dried to the extent necessary to form a solid material containing less than about 4% by weight of residual water.

After preparation of this solid anionic surfactant-containing material, this material can be combined with one or more of the non-aqueous organic diluents to form a structured, surfactant-containing liquid phase of the detergent compositions herein. This is done by reducing the anionic surfactant-containing material formed in the previously described pre-preparation step to powdered form and by combining such powdered material with an agitated liquid medium comprising one or more of the non-aqueous organic diluents, either surfactant or non-surfactant or both, as hereinbefore described. This combination is carried out under agitation conditions which are sufficient to form a thoroughly mixed dispersion of particles of the insoluble fraction of the co-dried LAS/salt material throughout a non-aqueous organic liquid diluent.

In a subsequent processing step, the non-aqueous liquid dispersion so prepared can then be subjected to milling or high shear agitation under conditions which are sufficient to provide a structured, surfactant-containing liquid phase of the detergent compositions herein. Such milling or high shear agitation conditions will generally include maintenance of a temperature between about 10° C. and 90° C., preferably between about 20° C. and 60° C.; and a processing time that is sufficient to form a network of aggregated small particles of the insoluble fraction of the anionic surfactant-containing powdered material. Suitable equipment for this purpose includes: stirred ball mills, co-ball mills (Fryma), colloid mills, high pressure homogenizers, high shear mixers, and the like. The colloid mill and high shear mixers are preferred for their high throughput and low capital and maintenance costs. The small particles produced in such equipment will generally range in size from about 0.4 to 2 microns. Milling and high shear agitation of the liquid/solids combination will generally provide an increase in the yield value of the structured liquid phase to within the range of from about 1 Pa to 8 Pa, more preferably from about 1 Pa to 4 Pa.

After formation of the dispersion of LAS/salt co-dried material in the non-aqueous liquid, either before or after such dispersion is milled or agitated to increase its yield value, the particulate material to be used in the detergent compositions herein can be added. Such components which can be added under high shear agitation include a silica or titanium dioxide elasticizing agent; particles of substantially all of an organic builder, e.g., citrate and/or fatty acid, and/or an alkalinity source, e.g., sodium carbonate, can be added while continuing to maintain this admixture of composition components under shear agitation. Agitation of the mixture is continued, and if necessary, can be increased at this point to form a uniform dispersion of insoluble solid phase particulates within the liquid phase.

After some or all of the foregoing solid materials have been added to this agitated mixture, the particles of the colored speckles and the highly preferred peroxygen bleaching agent can be added to the composition, again while the mixture is maintained under shear agitation. By adding the peroxygen bleaching agent material last, or after all or most of the other components, and especially after alkalinity source particles, have been added, desirable stability benefits for the peroxygen bleach can be realized. If enzyme prills are incorporated, they are preferably added to the non-aqueous liquid matrix last.

As a final process step, after addition of all of the particulate material, agitation of the mixture is continued for a period of time sufficient to form compositions having the requisite viscosity, yield value and phase stability characteristics. Frequently this will involve agitation for a period of from about 1 to 30 minutes.

In adding solid components to non-aqueous liquids in accordance with the foregoing procedure, it is advantageous to maintain the free, unbound moisture content of these solid materials below certain limits. Free moisture in such solid materials is frequently present at levels of 0.8% or greater. By reducing free moisture content, e.g., by fluid bed drying, of solid particulate materials to a free moisture level of 0.5% or lower prior to their incorporation into the detergent composition matrix, significant stability advantages for the resulting composition can be realized.

The compositions of this invention, prepared as hereinbefore described, can be used to form aqueous washing solutions for use in the laundering and bleaching of fabrics. Generally, an effective amount of such compositions is added to water, preferably in a conventional fabric laundering automatic washing machine, to form such aqueous laundering/bleaching solutions. The aqueous washing/bleaching solution so formed is then contacted, preferably under agitation, with the fabrics to be laundered and bleached therewith.

An effective amount of the liquid detergent compositions herein added to water to form aqueous laundering/bleaching solutions can comprise amounts sufficient to form from about 500 to 7,000 ppm of composition in aqueous solution. More preferably, from about 800 to 3,000 ppm of the detergent compositions herein will be provided in aqueous washing/bleaching solution.

Method

The use of the suspending aid has been found beneficial in the chemical stability of the pro-perfume within the detergent matrix, whether it being aqueous or non-aqueous. Accordingly, there is provided a the use of the suspending aid for stabilising the pro-perfume component within the detergent composition.

Also provided herein is a method for providing a delayed release of the benefit agent, preferably a perfume composition, which comprises the step of contacting the surface to be treated with a composition of the invention, and preferably thereafter contacting the treated surface with a material, preferably an aqueous medium like moisture or any other means susceptible of releasing the perfume from the composition.

By “surface”, it is meant any surface onto which the compound can deposit. Typical examples of such material are fabrics, hard surfaces such as dishware, floors, bathrooms, toilet, kitchen and other surfaces in need of a delayed release of a perfume such as that with litter like animal litter. Preferably, the surface is selected from a fabric, a tile, a ceramic; more preferably is a fabric.

By “delayed release” is meant release of the benefit agent (e.g perfume) over a longer period of time than by the use of the benefit agent (e.g., perfume) itself.

Where the carrier is a polymer or component which has been chemically reacted with a benefit agent like perfume, the release of the benefit agent which is entrapped or embedded within the reacted carrier, i.e. not chemically reacted, is released from the carried benefit agent composition by hydrolysis of the reacted carrier. Indeed, the hydrolysis of the “protective shell” made by the reacted carrier into the respective aldehyde and/or ketone on the one hand and the polymer on the other will gradually open the shell, thereby enabling release of the entrapped benefit agent.

Still in another aspect of the invention, there is provided the use of the product of the invention for the manufacture of a laundry and cleaning composition for delivering residual fragrance onto the fabrics on which it is applied.

For the purposes of the present invention the term “contacting” is defined as “intimate contact of a surface with an aqueous solution of the hereinabove described composition.” Contacting typically occurs by soaking, washing, rinsing the composition onto fabric, but can also include contact of a substrate inter alia a material onto which the composition has been absorbed, with the fabric.

I-Synthesis of Delta Damascone with Lupasol WF

In a reaction vessel of 2l, placed on a rotary evaporator, 1500 g of δ-Damascone and 1800 g of water-free Lupasol WF are mixed together for 4 hours at 42° C. at a stirring rate of 20 to 100 rpm. The temperature of the reaction mixture, during the mixing, is controlled by means of a water bath and not allowed to go higher than 42° C.; the temperature inside the reaction container is of 40° C. Subsequently, the reaction mixture is kept during 16 hours at 42° C. under vacuum to remove most of the water from the reaction vessel. 2741 g of product is obtained and only traces of unreacted δ-Damascone remain. The viscosity of the obtained pro-perfume component was of 10.000.000 cps.

II-Synthesis of Delta Damascone with Lupasol HF and Additional Perfume Composition

In a reaction vessel of 250 ml, 20 g of δ-Damascone and 16 g of Water-Free Lupasol HF (water-free Lupasol HF is taken from the commercial Lupasol sample from which the water has been removed by vaccum distillation) and 83 g of a perfume mixture are mixed together for 4 hours at 42° C. The temperature of the reaction mixture, during the mixing, is controlled via a thermostat and not allowed to go higher than 42° C. 118 g of product is obtained and only traces of unreacted δ-Damascone remain. The viscosity of the synthesised pro-perfume component is 1600 cps.

Any type of perfume mixture may be used. One preferred composition of the perfume mixture is as follows:

| | |
|---|---|
| Citronellol | 7 |
| Geraniol | 7 |
| Linalool | 7 |
| Para Tertiary Butyl Cyclohexyl Acetate | 10 |
| Phenyl Ethyl Alcohol | 19 |
| Habanolide | 4.5 |
| Para Methoxy Acetophenone | 1.5 |
| Benzyl Acetate | 4 |
| Eugenol | 2 |
| Phenyl Ethyl Acetate | 5 |
| Verdyl Acetate | 6 |
| Verdyl Propionate | 4 |
| Hexyl Cinnamic Aldehyde | 3 |
| Ionone Gamma Methyl | 2 |
| Methyl Cedrylone | 10 |
| P.T. Bucinal | 7 |
| Para Cresyl Methyl Ether | 1 |

Processing of the Pro-Perfume Composition

The synthesised pro-perfume I or II above mentioned is then mixed with either a silicone glycol ether DC3225C from Dow Corning or an hexamethyl disiloxane (linear silicone) DC 200 fluids with a viscosity of 100 cps measured at 20° C. from Dow Corning or a dimethylpolysiloxoxane with a viscosity of 60.000 cps from Sigma under high shear mixing (i.e. 50.000 rpm) using an Ultra Turrax apparatus at a temperature of 60° C. for 5 minutes. The pro-perfume component and the suspending aid are present in weight ratios of 50:50. The obtained suspension is then added as such to the liquid detergent matrix under low shear mixing (i.e. 200 rpm).

The obtained suspension may be subject to creaming on the top, i.e. exhibiting some physical separation. This can be overcome by the addition of Quartz droplets or by adjusting the density as conventionally known by the skilled person.

Abbreviations Used in the Following Detergent Composition Examples

In the detergent compositions, the abbreviated component identifications have the following meanings:

| | |
|---|---|
| LAS | Sodium linear alkyl benzene sulfonate |
| MEA | Monoethanolamine |
| PG | Propanediol |
| BPP | Butoxy-propoxy-propanol |
| EtOH | Ethanol |
| NaOH | Solution of sodium hydroxide |
| NaTS | Sodium toluene sulfonate |
| Citric | Anhydrous citric acid |
| acid | |
| CxyFA | C₁ₓ-C_{1y} fatty acid |
| TPKFA | C₁₂-C₁₄ topped whole cut fatty acids. |
| TFAA | C16-18 alkyl N-methyl glucamide |
| LMFAA | C12-14 alkyl N-methyl glucamide |
| APA | C8-C10 amido propyl dimethyl amine |
| CxyAS | Sodium C₁ₓ-C_{1y} alkyl sulfate (or other salt if |
| | specified) |
| CxyEzS | Sodium C₁ₓ-C_{1y} alkyl sulfate condensed |
| | with z moles of ethylene oxide (or other salt if |
| | specified) |
| CxyEz | A C_{1x-1y} branched primary alcohol condensed with |
| | an average of z moles of ethylene oxide |
| Nonionic | C13-C15 mixed ethoxylated/propoxylated fatty |
| | alcohol with average degree of ethoxylation of 3.8 and |
| | an average degree propoxylation of 4.5. |
| SCS | Sodium cumene sulphonate. |
| QAS | R₂.N + (CH₃)₂(C₂H₄OH) with R₂ = C₁₂-C₁₄. |
| TEPAE | Tetreaethylenepentaamine ethoxylate. |
| PAEC | methyl quaternized ethoxylated dihexylene triamine |
| MA/AA | Copolymer of 1:4 maleic/acrylic acid, average |
| | molecular weight about 70,000. |
| Protease | Proteolytic enzyme of activity 4 KNPU/g sold by |
| | NOVO Industries A/S under the tradename Savinase |
| | unless otherwise specified |
| Cellulase | Cellulytic enzyme of activity 1000 CEVU/g sold by |
| | NOVO Industries A/S under the tradename Carezyme |
| Amylase | Amylolytic enzyme of activity 60 KNU/g sold by |
| | NOVO Industries A/S under the tradename Termamyl |
| | 60T unless otherwise specified |
| Lipase | Lipolytic enzyme of activity 100 kLU/g sold by |
| | NOVO Industries A/S under the tradename Lipolase |
| | unless otherwise specified |
| Endolase | Endoglunase enzyme of activity 3000 CEVU/g sold by |
| | NOVO Industries A/S |
| PB1 | Anhydrous sodium perborate bleach of nominal |
| | formula NaBO₂.H₂O₂ |
| TAED | Tetraacetylethylenediamine |
| DTPA | Diethylene triamine pentaacetic acid |
| DTPMP | Diethylene triamine penta (methylene phosphonate), |
| | marketed by Monsanto under Trade name Dequest |
| | 2060 |
| Brightener 1 | Disodium 4,4′-bis(2-sulphostyryl)biphenyl |
| Brightener 2 | Disodium 4,4′-bis(4-anilino-6-morpholino-1.3.5- |
| | triazin-2-yl)amino) stilbene-2:2′-disulfonate. |
| HEDP | 1,1-hydroxyethane diphosphonic acid |
| EDDS | Ethylenediamine-N,N′-disuccinic acid, (S,S) isomer in |
| | the of its sodium salt |
| PVNO | Polyvinylpyridine-N-Oxide, with an average molecular w [symbol] |
| | w of 50,000. |
| SRP 1 | Sulfobenzoyl end capped esters with oxyethylene oxy |
| | and terephthaloyl backbone |
| Silicone | Polydimethylsiloxane foam controller with siloxane- |
| antifoam | oxyalkylene copolymer as dispersing agent with a ratio |
| | of said foam controller to said dispersing agent of 10:1 |
| | to 100:1. |
| CaCl2 | Calcium chloride |
| PPC1 | Pro-perfume composition as given from Synthesis |
| | Example I and mixed with a silicone glycol ether DC |
| | 3225C as above mentioned |
| PPC2 | Pro-perfume composition as given from Synthesis |
| | Example I and mixed with hexamethyl disiloxane DC |
| | 200 fluids or dimethylpolysiloxoxane with a viscosity |
| | of 60.000 cps as above mentioned |
| PPC3 | Pro-perfume composition as given from Synthesis |
| | Example II and mixed with a silicone glycol ether DC |
| | 3225C as above mentioned |
| PPC4 | Pro-perfume composition as given from Synthesis |
| | Example II and mixed with hexamethyl disiloxane DC |
| | 200 fluids as above mentioned |
| Polymer | Polyvinylpyrrolidone K90 available from BASF |
| | under the tradename Luviskol K90 |
| Dye fixative | Dye fixative commercially available from Clariant |
| | under the tradename Cartafix CB |
| Polyamine | 1,4-Bis-(3-aminopropyl)piperazine |
| Bayhibit AM | 2-Phosphonobutane-1,2,4-tricarboxylic acid |
| | commercially available from Bayer |
| Fabric | Di-(canoloyl-oxy-ethyl)hydroxyethyl methyl |
| softener | ammonium methylsulfate |
| active | |
| Genamin | Coco fatty amine ethoxylated with 10 moles ethylene |
| C100 | oxide and commercially available from Clariant |
| Genapol | Coco alcohol ethoxylated with 10 moles ethylene |
| V4463 | oxide and commercially available from Clariant |
| DEQA | Di-(tallowyl-oxy-ethyl) dimethyl ammonium chloride |
| DTDMAC | Ditallow dimethylammonium chloride |
| Fatty acid | Stearic acid of IV = 0 |
| Electrolyte | Calcium chloride |
| PEG | Polyethylene Glycol 4000 |
| Silicone | Polydimethylsiloxane foam controller with siloxane- |
| antifoam | oxyalkylene copolymer as dispersing agent with |
| | a ratio of said foam controller to said dispersing |
| | agent of 10:1 to 100:1. |

### EXAMPLE I

### Preparation of LAS Powder for Use as a Structurant

Sodium C₁₂ linear alkyl benzene sulfonate (NaLAS) is processed into a powder containing two phases. One of these phases is soluble in the non-aqueous liquid detergent compositions herein and the other phase is insoluble. It is the insoluble fraction which serves to add structure and particle suspending capability to the non-aqueous phase of the compositions herein.

NaLAS powder is produced by taking a slurry of NaLAS in water (approximately 40-50% active) combined with dissolved sodium sulfate (3-15%) and hydrotrope, sodium sulfosuccinate (1-3%). The hydrotrope and sulfate are used to improve the characteristics of the dry powder. A drum dryer is used to dry the slurry into a flake. When the NaLAS is dried with the sodium sulfate, two distinct phases are created within the flake. The insoluble phase creates a network structure of aggregate small particles (0.4-2 um) which allows the finished non-aqueous detergent product to stably suspend solids.

The NaLAS powder prepared according to this example has the following makeup shown in Table III.

TABLE I
LAS Powder

| Component | Wt. % |
|---|---|
| NaLAS | 85% |
| Sulfate | 11% |
| Sulfosuccinate | 2% |
| Water | 2.5% |
| Unreacted, etc. | balance to |
| | 100% |
| % insoluble LAS | 17% |
| # of phase (via X-ray | 2 |
| diffraction) | |

### EXAMPLE II

Non-aqueous based heavy duty liquid laundry detergent compositions (I to V) according to the present invention are presented below.

Non-Aqueous Liquid Detergent Composition with Bleach

| | | Wt % | Wt % | Wt % | |
|---|---|---|---|---|---|
| Component | Wt % I | II | III | IV | Wt % V |
| LAS, From Example I | 16 | 13 | 8 | 8 | 2 |
| C24E5 | 22 | 25 | 28 | 30 | 34 |
| BPP | 19 | 19 | 19 | 19 | 19 |
| Sodium citrate dihydrate | 3 | 3 | 3 | 3 | 3 |
| TAED | 5.9 | 5.9 | 5.9 | 5.9 | 5.9 |
| Sodium carbonate | 9 | 9 | 9 | 9 | 9 |
| MA/AA | 3 | 3 | 3 | 3 | 3 |
| Colored speckles | 0.4 | 0.4 | 0.4 | 0.4 | 0.4 |
| EDDS | 1 | 1 | 1 | 1 | 1 |
| Cellulase Prills | 0.1 | 0.1 | 0.1 | 0.1 | 0.1 |
| Amylase Prills | 0.4 | 0.4 | 0.4 | 0.4 | 0.4 |
| PAEC | 1.3 | 1.3 | 1.3 | 1.3 | 1.3 |
| PB1 | 15 | 15 | 15 | 15 | 15 |
| PPC1 | 0.4 | — | — | — | — |
| PPC2 | — | 0.4 | — | — | — |
| PPC3 | — | — | 0.4 | — | 0.2 |
| PPC4 | — | — | — | 0.4 | 0.2 |
| Optionals including: | balance | balance | balance | balance | balance |
| brightener, colorant, | | | | | |
| perfume, thickener, suds | | | | | |
| suppressor, colored | | | | | |
| speckles etc. | | | | | |
| | 100% | 100% | 100% | 100% | 100% |

The resulting Table II compositions are stable, anhydrous heavy-duty liquid laundry detergents which provide excellent stain and soil removal performance when used in normal fabric laundering operations whilst still providing good stability of the pro-perfume withn the detergent matrix as well as delayed release of the perfume onto the contacted fabric.

### EXAMPLE III

Liquid detergent compositions are made according to the following.

| | I | II | III | IV |
|---|---|---|---|---|
| C₂₅ E3S | 2 | 8 | 7 | 5 |
| LAS | 15 | 12 | 10 | 8 |
| C₁₂-C₁₄ alkyldimethyl amine oxide | — | — | — | 2 |
| C₂₅ AS | 6 | 4 | 6 | 8 |
| C₂₄ N-methyl glucamide | 5 | 4 | 3 | 3 |
| C₂₄ AE5 | 6 | 1 | 1 | 1 |
| C₂₈ FA | 11 | 4 | 4 | 3 |
| Citric acid | 1 | 3 | 3 | 2 |
| DTPMP | 1 | 1 | 1 | 0.5 |
| MEA | 8 | 5 | 5 | 2 |
| NaOH | 1 | 2.5 | 1 | 1.5 |
| PG | 14.5 | 13.1 | 10.0 | 8 |
| EtOH | 1.8 | 4.7 | 5.4 | 1 |
| Amylase (300 KNU/g) | 0.1 | 0.1 | 0.1 | 0.1 |
| Lipase D96/L (100 KNU/g) | 0.15 | 0.15 | 0.15 | 0.15 |
| Protease (35 g/l) | 0.5 | 0.5 | 0.5 | 0.5) |
| Endolase | 0.05 | 0.05 | 0.05 | 0.05 |
| Cellulase | 0.09 | 0.09 | 0.09 | 0.09 |
| SRP1 | 0.5 | — | 0.3 | 0.3 |
| Boric acid | 2.4 | 2.8 | 2.8 | 2.4 |
| Sodium xylene sulfonate | — | 3 | — | — |
| 2-butyl-octanol | 1 | 1 | 1 | 1 |
| silicone antifoam | 0.3 | 0.3 | 0.3 | 0.3 |
| PPC1 | 0.4 | — | 0.3 | 0.5 |
| PPC3 | — | 0.4 | — | — |

| | |
|---|---|
| Water & minors | Up to 100% |

The above liquid detergent compositions (A-D) are found to be very efficient in the removal of a wide range of stains and soils from fabrics under various usage conditions whilst still providing good stability of the pro-perfume withn the detergent matrix as well as delayed release of the perfume onto the contacted fabric.

### EXAMPLE IV

The following compositions (I to VI) are heavy duty liquid laundry detergent compositions according to the present invention.

| | Example #: | | | | | |
|---|---|---|---|---|---|---|
| | I | II | III | IV | V | VI |
| LAS | 17 | 15 | 7.0 | 7.0 | 12 | 12 |
| C35E3S/ | 2.0 | 9.0 | — | — | 7.0 | 7.0 |
| C25E3S | | | | | | |
| C25E2.5S | — | — | 12.0 | 12.0 | — | — |
| LMFAA | 6.0 | 5.0 | 4.5 | 3.7 | 4.0 | 4.0 |
| C35 E7 | 6.0 | 1.0 | — | — | — | — |
| C23 E9 | — | — | 2.0 | 1.0 | 5.0 | 5.0 |
| APA | — | 1.5 | — | 2.0 | — | 2.5 |
| C24 FA | 7.5 | 1.1 | 2.0 | 4.0 | 5.0 | 5.0 |
| C48 FA | 3.0 | 3.5 | — | — | — | — |
| Citric Acid | 1.0 | 3.5 | 3.0 | 3.0 | 3.0 | 3.0 |
| Protease | 0.6 | 0.6 | 0.9 | 0.9 | 1.2 | 1.2 |
| (34 g/#) | | | | | | |
| Lipase | 0.1 | 0.1 | 0.1 | 0.1 | 0.2 | 0.2 |
| Amylase | 0.1 | 0.1 | 0.1 | 0.1 | — | 0.1 |
| (300 KMU/g) | | | | | | |
| Cellulase | 0.03 | 0.03 | 0.05 | 0.05 | 0.2 | 0.2 |
| Endolase | 0.1 | 0.1 | — | — | — | — |
| Brightener 2 | 0.1 | 0.1 | — | — | — | — |
| Boric Acid | 3.0 | 3.0 | 3.5 | 3.5 | 4.0 | 4.0 |
| MEA | 8.0 | 4.0 | 1.0 | 1.5 | 7.0 | 7.0 |
| NaOH | 1.0 | 4.0 | 3.0 | 2.5 | 1.0 | 1.0 |
| PG | 12.0 | 12.0 | 7.5 | 7.5 | 7.0 | 7.0 |
| EtOH | 1.0 | 1.0 | 3.5 | 3.5 | 6.0 | 6.0 |
| Na TS | — | — | 2.5 | 2.5 | — | — |
| PPC2 | 0.4 | — | 0.1 | — | 0.3 | — |
| PPC4 | — | 0.5 | 0.3 | 0.4 | — | 0.2 |
| Minors | Balance | Balance | Balance | Balance | Balance | Balance |

### EXAMPLE V

The following non-aqueous liquid detergent compositions were prepared in accordance with the present invention:

| | I | II | III |
|---|---|---|---|
| LAS | 16.0 | 16.0 | 16.0 |
| C23 E5S | 21.5 | 21.5 | 19.0 |
| Butoxy Propoxy Propanol | 18.5 | — | 16.0 |
| Hexylene Glycol | — | 18.5 | 5.0 |
| Sodium citrate dihydrate | 6.8 | 6.8 | 3.8 |
| [4-[N-nonanoyl-6-aminohexanoyloxy] | 6.0 | 6.0 | 6.0 |
| benzene sulfonate] Na salt | | | |
| Methyl sulfate salt of methyl quaternized | 1.3 | 1.3 | 1.3 |
| polyethoxylated hexamethylene diamine | | | |
| EDDS | 1.2 | 1.2 | 1.2 |
| MA/AA | — | — | 3.0 |
| Sodium Carbonate | 10.0 | 10.0 | 10.0 |
| Protease | 0.05 | 0.02 | 0.02 |
| Amylase | 0.01 | 0.01 | 0.01 |
| Cellulase | 0.0001 | 0.0001 | 0.0001 |
| PB1 | 12.0 | 12.0 | 12.0 |
| Silicone antifoam | 0.75 | 0.75 | 1.1 |
| Perfume | 1.7 | 1.7 | 1.7 |
| Titanium Dioxide | 0.5 | 0.5 | 0.5 |
| Dichloro-5,12-Dimethyl-1,5,8,12- | — | 0.03 | 0.03 |
| tetraazabicyclo [6.6.2] hexadecane | | | |
| Manganese (II) | | | |
| Brightener 2 | 0.2 | 0.2 | 0.2 |
| Sodium hydrogenated C16-18 fatty soap | 1 | 1 | 0.5 |
| Colored Speckles | 0.4 | 0.4 | 0.4 |
| PPC1 | 0.4 | 0.4 | 0.4 |
| Miscellaneous up to 100% | | | |

### EXAMPLE VI

The following aqueous liquid laundry detergent compositions were prepared according to the present invention:

| | I | II | III | IV | V | VI |
|---|---|---|---|---|---|---|
| LAS | — | — | — | 1.0 | 2.0 | — |
| C25AS | 16.0 | 13.0 | 14.0 | 5.0 | — | 6.5 |
| C25E3S | 5.0 | 1.0 | — | 10.0 | 19.0 | 3.0 |
| C25E7 | 2.0 | 3.5 | — | 2.5 | 2.0 | 5.0 |
| TFAA | 5.0 | 4.5 | 4.5 | 6.5 | 4.0 | — |
| APA | 2.0 | 1.0 | — | 3.0 | — | 0.5 |
| QAS | — | — | 2.0 | — | 1.5 | — |
| TPKFA | 4.5 | 8.0 | 15.0 | — | 5.0 | 5.0 |
| Citric acid | 2.2 | 3.0 | — | 0.5 | 1.0 | 2.0 |
| Rapeseed | 2.0 | — | — | 3.0 | 6.0 | 1.5 |
| fatty acid | | | | | | |
| Ethanol | 3.2 | 2.0 | 2.5 | 2.2 | — | 0.5 |
| 1,2 Propanediol | 5.7 | 8.5 | 6.5 | 7.0 | 7.0 | 5.5 |
| Mono- | 5.0 | 7.5 | — | 5.0 | 1.0 | 2.0 |
| ethanolamine | | | | | | |
| TEPAE | — | 1.2 | — | 0.5 | 0.5 | — |
| DTPMP | — | 0.5 | — | 0.5 | — | 0.2 |
| HEDP | — | 0.5 | — | 1.0 | — | — |
| Protease | 0.02 | 0.03 | 0.02 | 0.02 | 0.02 | 0.01 |
| Lipase | 0.002 | 0.001 | 0.001 | — | 0.001 | — |
| Amylase | — | .0006 | — | — | 0.001 | — |
| Cellulase | 0.002 | 0.002 | — | 0.002 | 0.001 | — |
| SRP1 | 0.20 | 0.15 | 0.10 | — | 0.17 | 0.04 |
| PVNO | — | — | — | 0.05 | 0.10 | — |
| Brightener 1 | 0.20 | 0.15 | 0.10 | 0.05 | — | 0.05 |
| Suds | 0.25 | 0.20 | 0.15 | 0.15 | 0.30 | 0.10 |
| Suppressor | | | | | | |
| CaCl2 | 0.02 | 0.02 | — | 0.01 | 0.01 | — |
| Boric acid | 2.5 | 2.0 | 1.5 | 2.2 | 1.5 | 1.2 |
| Bentonite Clay | — | — | 5.5 | — | — | — |
| PPC1 | 0.4 | — | 0.2 | 0.4 | 0.3 | — |
| PPC3 | — | 0.4 | 0.1 | — | 0.1 | 0.2 |
| NaOH to pH | 8.0 | 7.5 | 7.7 | 8.0 | 7.0 | 7.5 |

Water/minors to 100%

### EXAMPLE VII

The following describe high density liquid detergent compositions according to the present invention:

weight %

| Ingredient | I | II | III | IV |
|---|---|---|---|---|
| Polyhydroxy Coco-Fatty Acid | 2.50 | 2.50 | — | — |
| Amide | | | | |
| C₂₃ AE₉ | — | — | 3.65 | 0.80 |
| C₂₅ AS | — | — | 6.03 | 2.50 |
| C₂₅ E_{1.8} S | 20.15 | 20.15 | — | — |
| C₄₅ E_{2.25} S | — | — | 18.00 | 18.00 |
| Alkyl N-Methyl Glucose Amide | — | — | 4.50 | 4.50 |
| C₁₀ Amidopropyl Amine | 0.50 | 0.50 | 1.30 | — |
| Citric Acid | 2.44 | 3.00 | 3.00 | 3.00 |
| C₂₄ FA | — | — | 2.00 | 2.00 |
| NEODOL 23-9¹ | 0.63 | 0.63 | — | — |
| Ethanol | 3.00 | 2.81 | 3.40 | 3.40 |
| Monoethanolamine | 1.50 | 0.75 | 1.00 | 1.00 |
| Propanediol | 8.00 | 7.50 | 7.50 | 7.00 |
| Boric Acid | 3.50 | 3.50 | 3.50 | 3.50 |
| Ethoxylated | 0.50 | — | — | — |
| tetraethylenepentamine² | | | | |
| Tetraethylenepentamine | — | 1.18 | — | — |
| Sodium Toluene Sulfonate | 2.50 | 2.25 | 2.50 | 2.50 |
| NaOH | 2.08 | 2.43 | 2.62 | 2.62 |
| Protease enzyme³ | 0.78 | 0.70 | — | — |
| Protease enzyme⁴ | — | — | 0.88 | — |
| ALCALASE⁵ | — | — | — | 1.00 |
| HEDP | 0.5 | 0.7 | 2.5 | 0.5 |
| Polyamine⁶ | 0.50 | 0.50 | — | — |
| PPC1 | 0.5 | 0.4 | 0.3 | 0.2 |
| Water⁷ | balance | balance | balance | balance |

¹Eg Ethoxylated Alcohols as sold by the Shell Oil Co.
²Ethoxylated tetraethylenepentamine (PEI 189 E₁₅-E₁₈) according to U.S. Pat. No. 4,597,898 Vander Meer issued Jul. 1, 1986.
³Bleach stable variant of BPN′ (Protease A-BSV) as disclosed in EP 130,756 A Jan. 9, 1985.
⁴Subtilisin 309 Loop Region 6 variant.
⁵Proteolytic enzyme as sold by Novo.
⁶Polyamine PEI 600 E20
⁷Balance to 100% can, for example, include minors like optical brightener, perfume, suds suppresser, soil dispersant, chelating agents, dye transfer inhibiting agents, additional water, and fillers, including CaCO₃, talc, silicates, etc.

### EXAMPLE VIII

The following liquid hard surface cleaning compositions were prepared according to the present invention:

| | A | B | C | D | E |
|---|---|---|---|---|---|
| PPC1 | 0.5 | — | 0.2 | — | 0.1 |
| PPC2 | — | 0.4 | — | 0.5 | 0.3 |
| Amylase | 0.01 | 0.002 | 0.005 | — | — |
| Protease | 0.05 | 0.01 | 0.02 | — | — |
| Hydrogen peroxide | — | — | — | 6.0 | 6.8 |
| Acetyl triethyl citrate | — | — | — | 2.5 | — |
| DTPA | — | — | — | 0.2 | — |
| Butyl hydroxy toluene | — | — | — | 0.05 | — |
| EDTA* | 0.05 | 0.05 | 0.05 | — | — |
| Citric/Citrate | 2.9 | 2.9 | 2.9 | 1.0 | — |
| LAS | 0.5 | 0.5 | 0.5 | — | — |
| C12 AS | 0.5 | 0.5 | 0.5 | — | — |
| C10AS | — | — | — | — | 1.7 |
| C12(E)S | 0.5 | 0.5 | 0.5 | — | — |
| C12, 13 E6.5 nonionic | 7.0 | 7.0 | 7.0 | — | — |
| Neodol 23-6.5 | — | — | — | 12.0 | — |
| Dobanol 23-3 | — | — | — | — | 1.5 |
| Dobanol 91-10 | — | — | — | — | 1.6 |
| C25E1.8S | — | — | — | 6.0 | |
| Na paraffin sulphonate | — | — | — | 6.0 | |
| Perfume | 1.0 | 1.0 | 1.0 | 0.5 | 0.2 |
| Propanediol | — | — | — | 1.5 | |
| Ethoxylated tetraethylene | — | — | — | 1.0 | — |
| pentaimine | | | | | |
| 2, Butyl octanol | — | — | — | — | 0.5 |
| Hexyl carbitol** | 1.0 | 1.0 | 1.0 | — | — |
| SCS | 1.3 | 1.3 | 1.3 | — | — |
| pH adjusted to | 7-12 | 7-12 | 7-12 | 4 | — |

| | |
|---|---|
| Miscellaneous and water | Up to 100% |

*Na4 ethylenediamine diacetic acid
**Diethylene glycol monohexyl ether

### EXAMPLE IX

The following spray composition for cleaning of hard surfaces and removing household mildew was prepared according to the present invention:

| | |
|---|---|
| PPC2 | 0.8 |
| Amylase | 0.01 |
| Protease | 0.01 |
| Na octyl sulfate | 2.0 |
| Na dodecyl sulfate | 4.0 |
| Na hydroxide | 0.8 |
| Silicate | 0.04 |
| Butyl carbitol* | 4.0 |
| Perfume | 0.35 |
| Water/minors | up to 100% |

*Diethylene glycol monobutyl ether

### EXAMPLE X

The following lavatory cleansing block compositions were prepared according to the present invention.

| | A | B | C |
|---|---|---|---|
| C16-18 fatty alcohol/50EO | 70.0 | — | — |
| LAS | — | — | 80.0 |
| Nonionic | — | 1.0 | — |
| Oleoamide surfactant | — | 25.0 | — |
| Partially esterified copolymer of vinylmethyl | 5.0 | — | — |
| ether and maleic anhydride, viscosity 0.1-0.5 | | | |
| Polyethylene glycol MW 8000 | — | 38.0 | — |
| Water-soluble K-polyacrylate MW 4000-8000 | — | 12.0 | — |
| Water-soluble Na-copolymer of acrylamide | — | 19.0 | — |
| (70%) and acryclic acid (30%) low MW | | | |
| Na triphosphate | 10.0 | — | — |
| Carbonate | — | — | — |
| PPC1 | 0.5 | — | 0.2 |
| PPC2 | — | 0.5 | 0.2 |
| Dye | 2.5 | 1.0 | 1.0 |
| Perfume | 3.0 | — | 7.0 |

| | |
|---|---|
| KOH/HCL solution | pH 6-11 |

### EXAMPLE XI

The following toilet bowl cleaning composition was prepared according to the present invention.

| | A | B |
|---|---|---|
| C14-15 linear alcohol 7EO | 2.0 | 10.0 |
| Citric acid | 10.0 | 5.0 |
| PPC1 | 0.5 | — |
| PPC2 | — | 0.5 |
| DTPMP | — | 1.0 |
| Dye | 2.0 | 1.0 |
| Perfume | 3.0 | 3.0 |

| | |
|---|---|
| NaOH | pH 6-11 |
| Water and minors | Up to 100% |

### EXAMPLE XII

The following fabric softening compositions are in accordance with the present invention

| Component | A | B | C | D | E | F |
|---|---|---|---|---|---|---|
| DTDMAC | — | — | — | — | 4.5 | 15.0 |
| DEQA | 2.6 | 2.9 | 18.0 | 19.0 | — | — |
| Fatty acid | 0.3 | — | 1.0 | — | — | — |
| HCl | 0.02 | 0.02 | 0.02 | 0.02 | 0.02 | 0.02 |
| PEG | — | — | 0.6 | 0.6 | — | 0.6 |
| Perfume | 1.0 | 1.0 | 1.0 | 1.0 | 1.0 | 1.0 |
| Silicone antifoam | 0.01 | 0.01 | 0.01 | 0.01 | 0.01 | 0.01 |
| PPC2 | 0.4 | 0.1 | 0.8 | 0.2 | 1.0 | 0.6 |
| Electrolyte (ppm) | — | — | 600 | 1200 | — | 1200 |
| Dye (ppm) | 10 | 10 | 50 | 50 | 10 | 50 |

Water and minors to balance to 100%

### EXAMPLE XIII

The following are non-limiting examples of pre-soak fabric conditioning and/or fabric enhancement compositions according to the present invention which can be suitably used in the laundry rinse cycle.

| Ingredients | A | B | C | D | E | F |
|---|---|---|---|---|---|---|
| Polymer | 3.5 | 3.5 | 3.5 | 3.5 | 3.5 | 3.5 |
| Dye fixative | 2.3 | 2.3 | 2.4 | 2.4 | 2.5 | 2.5 |
| Polyamine | 15.0 | 15.0 | 17.5 | 17.5 | 20.0 | 20.0 |
| Bayhibit AM | 1.0 | 1.0 | 1.0 | 1.0 | 1.0 | 1.0 |
| C₁₂-C₁₄ | — | 5.0 | 5.0 | — | — | — |
| dimethyl | | | | | | |
| hydroxyethyl | | | | | | |
| quaternary | | | | | | |
| ammonium | | | | | | |
| chloride | | | | | | |
| Fabric softener | — | — | 2.5 | 2.5 | — | — |
| active | | | | | | |
| Genamin C100 | 0.33 | — | 0.33 | 0.33 | 0.33 | — |
| Genapol V4463 | 0.2 | — | 0.2 | 0.2 | 0.2 | — |
| PPC2 | 0.5 | 1.0 | 0.2 | 0.8 | 0.1 | 0.4 |
| Water & | balance | balance | balance | balance | balance | balance |
| minors | | | | | | |

### EXAMPLE XIV

Liquid compositions A to H comprise from 30 wt % to 40 wt % water

| Ingredient | A | B | C | D | E | F | G | H |
|---|---|---|---|---|---|---|---|---|
| APA | 1.5 | | 1 | 2 | 1.5 | 1 | 1.3 | 1.2 |
| CFAA | 5 | 4 | 3 | 4 | | | 4 | |
| C28AS | 15 | | | | | | 12 | 17 |
| C24E7 | 5 | | 2 | | 3 | 5 | 4.5 | 2.5 |
| C25E1.25S | | | 20 | 10 | 20 | 15 | | |
| C25E3S | 5 | 15 | | | | | 4 | |
| HLAS | | | | | 5 | 4 | | 5 |
| QAS(1) | | 1.5 | | | | | | |
| Boric acid(1) | | 1 | | | | | | |
| Boric acid(2) | | | 0.2 | 0.15 | 0.15 | 0.2 | 2 | 2 |
| Ethanol | 1.5 | 3.5 | 3 | 1 | 1 | 2 | 1 | 3 |
| Hydroxide | | 6.5 | | | 1.5 | 1.5 | | 2 |
| MEA | 10 | | 7 | 8 | 7 | 9 | 9 | 5 |
| Propanediol | 9 | 7 | 7 | 8 | 8 | 9 | 8 | 9 |
| Citric Acid | 1 | 1.5 | 2 | 0.5 | 2.5 | 1 | 1 | 2.5 |
| RFA | 3 | | 5 | 1 | 3 | 3 | 2.5 | 4.5 |
| TPKFA | 7.5 | 17 | 3 | 8 | 5 | 4 | 7.5 | 3 |
| DTPMP | 1 | 1.5 | 1 | 0.3 | | 0.5 | 0.6 | 1 |
| HEDP | 0.3 | | 0.3 | | 0.5 | 0.35 | | |
| Brightener(2) | 0.1 | 0.15 | 0.2 | 0.1 | 0.15 | 0.2 | | |
| PVNO | | | | | | | 0.05 | 0.1 |
| Clay | | 4 | | | | | | |
| SRP(2) | 0.1 | | 0.15 | | 0.1 | | | 0.15 |
| Amylase | | 0.1 | 0.0 | 0.1 | 0.7 | 0.3 | 0.15 | 0.2 |
| Cellulase | | | | 0.01 | 0.1 | 0.3 | 0.01 | 0.2 |
| Lipase | | | | 0.05 | | | | 0.06 |
| Mannanase | | | | 0.3 | 0.05 | | 0.06 | |
| Protease | | 0.5 | 0.7 | 0.6 | 0.5 | 0.4 | 0.7 | 0.6 |
| Perfume | 0.5 | | | 1.0 | | | 0.3 | |
| Silicone | 0.2 | 0.2 | 0.2 | 0.1 | 0.1 | 0.2 | 0.2 | 0.2 |
| antifoam | | | | | | | | |
| PPC1, PPC2, | 0.5 | 0.4 | 2.1 | 0.6 | 0.5 | 0.6 | 0.6 | 3.5 |
| PPC3 and/or | | | | | | | | |
| PPC4 | | | | | | | | |
| Miscellaneous | to | to | to | to | to | to | to | to |
| | 100% | 100% | 100% | 100% | 100% | 100% | 100% | 100% |

### EXAMPLE XV

Liquid compositions A to H comprise from 30% wt to 40 wt % water:

| Ingredient | A | B | C | D | E | F | G | H |
|---|---|---|---|---|---|---|---|---|
| APA | 1 | | | 1.5 | 1 | 1 | 2 | 1.5 |
| C23E9 | 2.5 | 2 | 3 | 1 | 3.5 | 2.5 | | |
| C25E1.1S | 15 | 20 | 18 | 17 | 25 | 20 | 17 | 19 |
| HLAS | | 2.5 | 2 | 3 | 1.5 | 2 | 2 | |
| Boric acid(2) | 2.5 | 2 | 2.5 | 1.5 | 2 | 1.5 | 2 | 2 |
| Ethanol | 1.5 | 2.5 | 2 | 2 | 3 | 4 | 3 | 1 |
| Hydroxide | 2 | 3 | 3 | 2 | 3 | 2 | 3 | 3 |
| MEA | 3 | 3 | 2 | 2 | 2.5 | 3 | 2 | 3 |
| Propanediol | 5 | 6 | 6 | 5 | 5 | 6 | 15 | 10 |
| Citric Acid | 3 | 2.5 | 2 | 3 | 3 | 2.5 | 3.5 | 3 |
| TPKFA | 4 | 4 | 3 | 5 | 2 | 3 | 4 | 3 |
| Brightener(2) | 0.2 | 0.1 | 0.15 | 0.15 | 0.2 | 0.2 | 0.1 | 0.1 |
| Amylase | 0.1 | 0.05 | 0.2 | 0.1 | 0.15 | | 0.05 | |
| Cellulase | 0.05 | 0.1 | 0.06 | 0.01 | 0.05 | 0.15 | 0.07 | |
| Mannanase | 0.1 | | 0.05 | 0.15 | 0.1 | 0.1 | | |
| Protease | 0.6 | 0.9 | 0.1 | 1.2 | 0.8 | 0.5 | 1 | |
| Perfume | 0.5 | | | 0.2 | 0.05 | | | |
| Silicone | 0.01 | | 0.02 | 0.01 | 0.02 | | | |
| antifoam | | | | | | | | |
| PPC, PPC2, | 0.4 | 2.5 | 0.6 | 0.2 | 0.1 | 3.3 | 0.5 | 0.8 |
| PPC3 and/or | | | | | | | | |
| PPC4 | | | | | | | | |
| Miscellaneous | to | to | to | to | to | to | to | to |
| | 100% | 100% | 100% | 100% | 100% | 100% | 100% | 100% |

What is claimed is:

1- A pro-perfume composition comprising a suspending material, a pro-perfume component comprising an amino-functional component and a benefit agent, characterised in that the pro-perfume component and the suspending material are in close physical proximity.

2- A composition according to claim 1, wherein the amino-functional component is selected from ethyl-4-amino benzoate, polyethyleneimine polymers; diaminobutane dendrimers Astramol®, polylysine, cross-linked polylysine, N,N′-bis-(3-aminopropyl)-1,3-propanediamine linear or branched; 1,4-bis-(3-aminopropyl) piperazine, and mixtures thereof.

3- A composition according to either one of claim 1 or 2, wherein the benefit agent is a perfume, preferably selected from a ketone perfume ingredient, aldehyde perfume ingredient, and mixture thereof.

4- A composition according to any one of claim 1-3, wherein the perfume ingredient is a ketone perfume ingredient, preferably selected from Alpha Damascone, Delta Damascone, Iso Damascone, Carvone, Gamma-Methyl-Ionone, Iso-E-Super, 2,4,4,7-Tetramethyl-oct-6-en-3-one, Benzyl Acetone, Beta Damascone, Damascenone, methyl dihydrojasmonate, methyl cedrylone, hedione, and mixtures thereof.

5- A composition according to any one of claim 1-4, wherein the perfume ingredient is an aldehyde perfume ingredient, preferably selected from citral, 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P.T. Bucinal, lyral, cymal, methyl nonyl acetaldehyde, trans-2-nonenal, lilial, trans-2-nonenal, and mixture thereof.

6- A composition according to any one of claim 1-5, wherein the ketone and/or aldehyde ingredient are reacted with the amino-functional component.

7- A composition according to any one of claim 1-6, wherein the pro-perfume composition further comprises an additional perfume composition.

8- A composition according to any one of claims 1-7, wherein the suspending material is hydrophobic, and preferably has a viscosity, as measured with on a rheometer, TA Instrument CSL²₁₀₀ at a temperature of 20° C. with a gap setting of 500 microns, between 250 and 250000 cps.

9- A composition according to any one of claim 1-8, wherein the suspending material is a silicone, or a mixture of silicones, preferably selected from cyclic silicones; polydimethyl siloxane gums and fluids; cyclic silicone fluid of the formula [(CH₃)₂SiO]ₙ where n ranges between about 3 to about 7, linear silicone polymer fluid having the formula (CH₃)₃SiO[(CH₃)₂SiO]ₘSi(CH₃)₃ where m can be 0 or greater and has an average value such that the viscosity at 25° C. of the silicone fluid is preferably 5 centistokes or more, fluorosilicones, and/or silicone surfactants such as silicone polyglycolethers.

10- A composition according to claim 9 whereby the suspending material comprises a silicone, fluorosilicone and/or silicone surfactant having a weight average molecular weight of more than 1000, preferably more than 2500.

11- A composition according to claim 8 or 9 whereby the suspending material comprises a silicone surfacant having a HBL value of 5 or less, preferably 3 or less.

12- A composition according to any one of claims 1-10, wherein the suspending material comprises a material selected from benzyl salicylate, diethyl phatalate, dipropylene glycol, butoxypropoxypropanol, and mixtures thereof, more preferably is a mixture comprises at least a silicones, preferably a mixture of silicones.

13- A process for making the pro-perfume composition as defined in claim 1-8, which comprises the steps of mixing with high shear the pro-perfume and the suspending material.

14- Pro-perfume composition as obtainable from the process of claim 11, preferably having a particle size between 1 to 50 microns, more preferably between 2 and 20 or even 15 microns.

15- A detergent composition comprising one or more detergent ingredients and a pro-perfume composition as defined in any one of claim 1-10 or 12.

16- A detergent composition according to claim 13, wherein said composition is a liquid detergent composition, preferably selected from aqueous liquid detergent composition and non-aqueous liquid detergent compositions.

17- A detergent composition according to either one of claim 13 or 14, wherein said pro-perfume composition is incorporated by mixing the pro-perfume composition with the detergent ingredient.

18- Use of a suspending aid as defined in claim 1 or 8-10 for improving the chemical stability of the pro-perfume component defined in any one of claim 1-12 within a liquid detergent matrix.

19- A method for providing a long lasting release of the perfume, which comprises the steps of contacting the fabric with a composition as defined in any one of claim 1-15.

20- A method according to claim 17, wherein the contacting occurs during the wash cycle.
